(19) United States
(12) Patent Application Publication
(10) Pub. No.: US 2024/0287420 A1
(43) Pub. Date: Aug. 29, 2024

(54) COMPOSITIONS CONTAINING COLOUR-NEUTRAL DEGRADABLE MICROCAPSULES

(71) Applicant: HENKEL AG & CO. KGAA, Düsseldorf (DE)

(72) Inventors: Andreas BAUER, Kaarst (DE); Marc-Steffen SCHIEDEL, Monheim (DE); Stefan URLICHS, Essen (DE); Manuela MATERNE, Kaarst (DE); Hubert SMYREK, Krefeld (DE); Christian GIESEN, Nettetal (DE); Andreas GERIGK, Erkelenz (DE); Christian KIND, Baden-Baden (DE); Claudia MEIER, Lichtenau (DE); Jeanette HILDEBRAND, Bühl (DE); Michael BIEDENBACH, Oberkirch (DE)

(21) Appl. No.: 18/568,177

(22) PCT Filed: Jun. 10, 2022
(86) PCT No.: PCT/EP2022/065834

§ 371 Date: Dec. 7, 2023

(30) Foreign Application Priority Data
Jun. 11, 2021 (EP) 21179039.9
Jun. 11, 2021 (EP) 21179043.1

(51) Int. Cl. C11D 3/50; A61K 8/11; A61Q 13/00

(52) U.S. Cl. CPC C11D 3/505; A61K 8/11; A61Q 13/00; A61K 2800/63; A61K 2800/70; A61K 2800/805

(57) ABSTRACT

Compositions selected from detergents and cleaning agents and cosmetic agents are described. The compositions include biodegradable microcapsules including a core material and a shell. The shell includes at least one barrier layer and one stability layer. The barrier layer surrounds the core material. The stability layer includes at least one biopolymer, and is arranged on the outer surface of the barrier layer. An emulsion stabilizer is arranged at the transition between the barrier layer and the stability layer. Use of these agents is also described.

### FIELD OF THE INVENTION

The invention relates to detergents, cleaning agents, and cosmetics which contain biodegradable microcapsules having environmentally compatible wall materials.

### BACKGROUND OF THE INVENTION

Microencapsulation is a versatile technology. It offers solutions for numerous innovations, from the paper industry to household products, to increase the functionality of a wide range of active substances. Encapsulated active substances can be used more economically and improve the sustainability and environmental compatibility of many products.

However, the polymer wall materials of the microcapsules themselves are environmentally compatible to very different degrees. Microcapsule walls that are based on the natural product gelatin and that are therefore completely biodegradable have been used for a long time in non-carbon paper. A method developed in the 1950s for gelatin encapsulation is disclosed in U.S. Pat. No. 2,800,457. A plurality of variations with respect to materials and method steps have since been described. In addition, biodegradable or enzymatically degradable microcapsule walls are used to utilize the enzymatic degradation as a method for releasing the core material. Microcapsules of this kind are described, for example, in WO 2009/126742 A1 or WO 2015/014628 A1.

However, microcapsules of this kind are not suitable for many industrial applications and household products. This is because natural-based microcapsules do not provide the diffusion resistance required for detergents, cleaning agents, adhesive systems, paints, and dispersions, nor do they provide the chemical resistance, the temperature resistance, or the required loading with core material.

In these so-called high-demand areas, organic polymers such as melamine-formaldehyde polymers (see, for example, EP 2 689 835 A1, WO 2018/114056 A1, WO 2014/016395 A1, WO 2011/075425 A1, or WO 2011/120772 A1); polyacrylates (see, for example, WO 2014/032920 A1, WO 2010/79466 A2); polyamides; polyurethane, or polyureas (see, for example, WO 2014/036082 A2 or WO 2017/143174 A1) are traditionally used. The capsules made from such organic polymers have the required diffusion tightness, stability, and chemical resistance. However, these organic polymers are enzymatically or biologically degradable only to a very small extent.

Various approaches have been described in the prior art in which biopolymers are combined as an additional component with the organic polymers of the microcapsule shell for use in high-demand areas, but not with the aim of preparing biodegradable microcapsules, but primarily the release, stability, or surface properties to modify the microcapsules. For example, WO 2014/044840 A1 describes a method for preparing two-layer microcapsules having an inner polyurea layer and an outer gelatin-containing layer. In this case, the polyurea layer is produced by means of polyaddition on the inside of the gelatin layer obtained by coacervation. According to the description, the capsules obtained in this manner have the necessary stability and tightness for use in detergents and cleaning agents, and additionally have a stickiness on account of the gelatin in order to adhere to surfaces. Specific stabilities and resistances are not mentioned. A disadvantage of polyurea capsules, however, is the unavoidable side reaction of the core materials with the diisocyanates used to produce the urea, which diisocyanates must be admixed with the oil-based core.

Moreover, microcapsules based on biopolymers are also described in the prior art, which achieve improved tightness or stability with respect to environmental influences or a targeted adjustment of a delayed release behavior by means of the addition of a protective layer. For example, WO 2010/003762 A1 describes particles having a core-shell-shell structure. In the interior of each particle, the core is an organic active ingredient that is poorly soluble or insoluble in water. The shell directly enclosing the core comprises a biodegradable polymer and the outer shell comprises at least one metal or metalloid oxide. A biodegradable shell is obtained with this structure, and according to WO 2010/003762 A1, the microcapsules are used in food, cosmetics, or pharmaceutical agents, however they cannot be used in the high-demand areas according to the invention due to a lack of tightness.

In the non-published document PCT/EP2020/085804, microcapsules having a multi-layer shell structure are described, the shells being substantially biodegradable and nevertheless having sufficient stability and tightness in order to be usable in high-demand areas. This is achieved by virtue of the fact that a stability layer makes up the majority of the capsule shell, which consists of naturally occurring and readily biodegradable materials, in particular gelatin or alginate or materials which are ubiquitously present in nature.

This stability layer is combined with a barrier layer, which can consist of known materials used for microencapsulation, such as melamine formaldehyde or meth(acrylate). In this case, it is possible to design the barrier layer to have a small wall thickness that was previously not feasible and nevertheless to ensure sufficient tightness. The proportion of the barrier layer in the overall wall is thus kept very low, such that the microcapsule wall has a biodegradability of at least 40%, measured according to OECD 301 F.

### SUMMARY OF THE INVENTION

It has been found that microcapsules with a multilayer structure of the shell formed of easily biodegradable (external) stability layer and thin (inner) barrier layer can be improved by the use of emulsion stabilizers after production of the inner barrier layer. Although emulsion stabilizers are regularly used for stabilizing the core material emulsion, it has been found, surprisingly, that treatment of the surface of the barrier layer enclosing the core material with an emulsion stabilizer, in particular a copolymer containing certain acrylic acid derivatives, leads to an improved deposition of the stability layer and thus to a larger average layer thickness of the stability layer (see Examples 2 to 4). Such microcapsules are particularly suitable for use in detergents and cleaning agents and also cosmetic preparations. It has also been found that these microcapsules are suitable in particular for the encapsulation of perfume compositions, as described herein.

According to the first aspect, the invention consequently relates to an agent selected from detergents and cleaning agents and cosmetic preparations, said agent containing biodegradable microcapsules comprising a core material and a shell, the shell consisting of at least one barrier layer and one stability layer, the barrier layer surrounding the core material, the stability layer comprising at least one biopolymer, and being arranged on the outer surface of the barrier layer, and an emulsion stabilizer being arranged at the transition between the barrier layer and the stability layer.

In various embodiments, the barrier layer and stability layer differ in terms of their chemical composition or their chemical structure. The core material preferably comprises at least one fragrance and may be, for example, a perfume (oil) composition.

The terms “perfume oil composition” and “perfume composition” can be used synonymously in the context of this invention.

In various embodiments, the core material comprises at least one perfume composition, wherein the perfume composition comprises, based on the total weight of all odorants contained in the perfume composition:
a) ≤10 wt. % of odorants having a CLogP of ≤2.5 and a boiling point of ≥200° C.;
b) ≥15 wt. % of at least one odorant having a CLogP of ≥4.0 and a boiling point of ≤275° C.; and
c) ≥30 wt. % of at least one odorant having a vapor pressure of ≥5 Pa at 20° C.

Furthermore, the perfume composition can comprise, in various embodiments, based on the total weight of all odorants contained in the perfume composition:

a) ≤8 wt. % of odorants having a CLogP of ≤2.5 and a boiling point of ≥200° C.;
b) ≥20 wt. % of at least one odorant having a CLogP of ≥4.0 and a boiling point of ≤275° C.; and/or
c) ≥40 wt. % of at least one odorant having a vapor pressure of ≥5 Pa at 20° C.

If the agent is a detergent or cleaning agent, it preferably contains at least one further component selected from surfactants, builders, enzymes, and attachment-enhancing agents. If the agent is a cosmetic agent, it may also contain at least one further component which can be selected, for example, from surfactants and skin care substances.

The emulsion stabilizer is a polymer or copolymer which is composed of particular acrylic acid derivatives, n-vinylpyrrolidone, and/or styrene. In various embodiments, the polymer or copolymer consists of one or more monomers selected from:
(1) acrylic acid derivatives of the general formula (I)

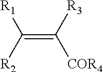

where
R₁, R₂, and R₅ are selected from hydrogen and an alkyl group having 1 to 4 carbon atoms, R₁ and
R₂ in particular being hydrogen, and R₃ in particular being hydrogen or methyl; and
R₄ representing —OX or —NR₅R₆, X representing hydrogen, an alkali metal, an ammonium group, or a C1-C18 alkyl optionally substituted by —SO₃M, or —OH, M being hydrogen, an alkali metal, or ammonium, the C1-C18 alkyl optionally substituted by —SO₃M or —OH preferably being methyl, ethyl, n-butyl, (2-)ethylhexyl, 2-sulfoethyl, or 2-sulfopropyl, R₅ and R₆ representing, independently of one another, hydrogen or a C1-C10 alkyl optionally substituted by —SO₃M, at least one of R₅ and R₆ not being hydrogen, R₅ preferably being H, and R₆ preferably being 2-methyl-propan-2-yl-1-sulfonic acid;
(2) N-vinylpyrrolidone, and
(3) styrene.

In the above embodiment, R₄ in various embodiments can represent —OX, X being hydrogen, C₁₋₁₀ alkyl, an alkali metal, or an ammonium group, preferably C₁₋₁₀ alkyl, more preferably methyl, n-butyl, or ethylhexyl, in particular 2-ethylhexyl.

The term “ethylhexyl” typically comprises or refers to 2-ethylhexyl and/or 3-ethylhexyl, preferably 2-ethylhexyl.

The emulsion stabilizer is preferably an acrylate copolymer comprising 2-acrylamido-2-methylpropanesulfonic acid (AMPS). A suitable copolymer is available, for example, under the trade name Dimension PA 140.

In various embodiments, the barrier layer is composed of one or more components selected from the group consisting of an aldehyde component, an aromatic alcohol, an amine component, an acrylate component, and an isocyanate component, and the stability layer comprises at least one biopolymer.

Furthermore, it is advantageous that the improved structural accommodation of the stability layer by the barrier layer by means of the addition of the emulsion stabilizer ensures the structural (covalent) bonding of all wall-forming components, and therefore the individual layers can be inseparably connected and regarded as a monopolymer.

Due to the robustness or tightness of the biodegradable capsule, it can be used in a large number of products from the field of detergents and cleaning agents and also cosmetics. A further advantage of the described microcapsules is the bright coloration of dispersions of these microcapsules. In various embodiments, the dispersions containing biodegradable microcapsules as described herein have a spectral locus with an L* value of at least 50 in the L*a*b* color space.

Furthermore, in a further aspect, the invention relates to the use of detergents and cleaning agents according to the first aspect in a method for conditioning textiles or for cleaning textiles and/or hard surfaces.

Furthermore, in a further aspect, the invention relates to the cosmetic use of agents according to the first aspect.

### BRIEF DESCRIPTION OF THE DRAWINGS

FIG. 1 is a photomicrograph of the various microcapsule dispersions in each case on the left in a 50× and 500× magnification recorded with an Olympus BX 50 microscope.
A) microcapsule dispersion slurry 2 according to the invention
B) microcapsule dispersion slurry 3 according to the invention
C) microcapsule dispersion slurry 5 according to the invention
D) microcapsule dispersion slurry 6 according to the invention
E) reference microcapsule dispersion MC1
F) reference microcapsule dispersion MC2
G) reference microcapsule dispersion MC4

FIG. 2 shows two microscopic images of the microcapsule dispersions slurry 2 and MC1 with auxiliary lines for determining the thickness of the stability layer of the microcapsules and information relating to the measured thicknesses.

FIG. 3 shows a diagram of the course of the biological degradation according to OECD 301 F over 60 days after washing of the microcapsule dispersions and references according to the invention. Slurry 2 and slurry 3 and the reference microcapsule dispersion MC1. In A), the degradation of slurry 2 and slurry 3 and additionally as positive controls of the degradation of ethylene glycol as well as of walnut shell flour is shown. In B) slurry 2 and slurry 3 with comparison with the reference capsule MC1 are shown.

FIG. 4 shows a diagram of the storage stability of various microcapsule dispersions as described in Example 5. A) Stability of the microcapsule dispersions slurry 2 and slurry 3 during storage over a period of 4 weeks in comparison with the reference capsule MC2. B) Stability of the microcapsule dispersion slurry 2 during storage over a period of 4 weeks in comparison with the reference capsules MC1 and MC2. C) Stability of the microcapsule dispersion slurry 3 during storage over a period of 4 weeks in comparison with the reference capsules MC2 and MC4. D) Stability of the microcapsule dispersion slurry 5 and slurry 6 in comparison with the reference capsule MC2.

FIG. 5 shows the diagram of the development of the L*a*b* of the microcapsule dispersions slurry 3 and 3A over a period of 8 days. A) Temporal development of the L* value. B) Temporal development of the a* value. C) Temporal development of the b* value.

### DETAILED DESCRIPTION OF THE INVENTION

### Definitions

“Barrier layer” refers to the layer of a microcapsule wall which is substantially responsible for sealing the capsule shell, i.e. prevents the core material from escaping.

“Biodegradability” refers to the ability of organic chemicals to be decomposed biologically, i.e. by living organisms or the enzymes thereof. Ideally, this chemical metabolism proceeds all the way up to mineralization, but may also stop at non-degradable transformation products. The OECD guidelines for testing chemicals, which are also used within the framework of the chemical approval process, are generally recognized. The tests of OECD test series 301 (A-F) show rapid and complete biological degradation (ready biodegradability) under aerobic conditions. Different test methods are available for highly or poorly soluble and for volatile substances. In particular, the manometric respiratory test (OECD 301 F) is used in the context of the application. The inherent biodegradability can be determined using the measurement standard OECD 302, for example the MITI Il test (OECD 302 C).

Within the context of the present invention, “biodegradable” or “biologically degradable” refers to microcapsule walls which have a biodegradability of at least 40% within 60 days, measured according to OECD 301 F. From a limit value of at least 60% degradation within 60 days measured according to OECD 301 F, microcapsule walls are also referred to as being rapidly biodegradable in the present case.

A “biopolymer” is a naturally occurring polymer, for example a polymer occurring in a plant, a fungus, a bacterium, or an animal. Biopolymers also include modified polymers based on naturally occurring polymers. The biopolymer can be obtained from the natural source or produced artificially.

“Tightness” relative to a substance, gas, liquid, radiation, or the like, is a property of material structures. The terms “tightness” and “sealing” are used synonymously according to the invention. Tightness is a relative term and is always based on predetermined framework conditions.

“Emulsion stabilizers” are auxiliary substances for stabilizing emulsions. The emulsion stabilizers can be added in a small amount to the aqueous or oily phase (of emulsions), said emulsion stabilizers being phase-enriched in the interface and, on the one hand, facilitate the separation of the internal phase by lowering the interfacial tension and, on the other hand, increase the separation resistance of the emulsion.

The term “(meth)acrylate” in this invention refers both to methacrylates and acrylates.

According to the invention, the term “microcapsules” should be understood to mean particles which contain an inner space or core which is filled with a solid, gelled, liquid, or gaseous medium and which is surrounded (encapsulated) by a continuous casing (shell) of film-forming polymers. These particles preferably have small dimensions. The terms “microcapsules”, “core-shell capsules”, or simply “capsules” are used synonymously.

“Microencapsulation” refers to a preparation method in which small and very small portions of solid, liquid, or gaseous substances are surrounded by a coating consisting of polymer or inorganic wall materials. The microcapsules obtained in this manner can have a diameter of from a few millimeters down to less than 1 μm. However, it may be preferred for the diameter to be greater than 100 nm or greater than 500 nm.

The microcapsule according to the invention has a multilayer “shell”. The shell encasing the core material of the microcapsule is also regularly referred to as “wall” or “envelope”.

The microcapsules according to the invention with a multilayer shell can also be referred to as multilayer microcapsules or multilayer microcapsule system, since the individual layers can also be regarded as individual shells. “Multi-layered” and “having multiple shells” are therefore used synonymously.

“Stability layer” refers to the layer of a capsule wall which is substantially responsible for the stability of the capsule shell, i.e. it generally makes up the majority of the shell.

“Wall formers” are the components that build up the microcapsule wall.

### Microcapsules

The biodegradable microcapsules which are used according to the first aspect of the invention in detergents, cleaning agents, and cosmetic agents comprise a core material and a shell, the shell consisting of at least one barrier layer and a stability layer, the barrier layer surrounding the core material, the stability layer comprising at least one biopolymer and being arranged on the outer surface of the barrier layer, and an emulsion stabilizer being arranged at the transition from the barrier layer to the stability layer. This arrangement may consist of an intermediate layer of emulsion stabilizer, which may be continuous or discontinuous and may cover parts of or the entire inner barrier layer. Alternatively, only individual molecules of the emulsion stabilizer may be arranged on the surface of the barrier layer such that they mediate a bond between the stability layer and the barrier layer. The emulsion stabilizer here acts as a mediator agent.

As shown in Example 4, the microcapsule shells according to the invention have a significantly increased thickness of the stability layer due to the use of the emulsion stabilizer. As a result, the proportion of natural components in the capsule is further increased compared to previously described multilayer microcapsules.

According to one embodiment of the biodegradable microcapsules, during preparation of the microcapsules, the surface of the barrier layer is brought into contact with the emulsion stabilizer before the stability layer is formed. As a result, the capacity of the surface to structurally bond the stability layer is increased. Without wishing to be limited thereto, the inventors assume that the emulsion stabilizer accumulates on the non-polar surface of the barrier layer, in particular a melamine-formaldehyde layer, and thus provides the biopolymers of the stability layer with a framework for deposition on the surface. As a result, not only is the mean layer thickness of the stability layer produced with the biopolymer increased, the emulsion stabilizer is also incorporated at the interface between the stability layer and the barrier layer. Proceeding from this theory, any emulsion stabilizer is, in principle, suitable as a mediator agent for preparing the microcapsules.

In a preferred embodiment, the emulsion stabilizer is a polymer or copolymer consisting of one or more monomers selected from:
(1) acrylic acid derivatives of the general formula (I)

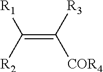

where
R₁, R₂, and R₅ are selected from hydrogen and an alkyl group having 1 to 4 carbon atoms, R₁ and
R₂ in particular being hydrogen, and R₃ in particular being hydrogen or methyl; and
R₄ representing —OX or —NR₅R₆, X representing hydrogen, an alkali metal, an ammonium group, or a C₁₋₁₈ alkyl optionally substituted by —SO₃M or —OH, for example C₁₋₁₀ alkyl, M being hydrogen, an alkali metal, or ammonium, the C₁₋₁₈ alkyl optionally substituted by —SO₃M or —OH, for example C1-C10 alkyl, preferably being methyl, ethyl, n-butyl, (2-)ethylhexyl, 2-sulfoethyl, or 2-sulfopropyl, R₅ and R₆ representing, independently of one another, hydrogen or a C₁₋₁₀ alkyl optionally substituted by —SO₃M, at least one of R₅ and R₆ not being hydrogen, R₅ preferably being H, and R₅ preferably being 2-methyl-propan-2-yl-1-sulfonic acid;
(2) N-vinylpyrrolidone, and
(3) styrene.

The C1-4 hydroxyalkyl groups possible for R₁, R₂, and R₃ may be ethyl, n-propyl, i-propyl, and n-butyl. In some embodiments of the acrylic acid derivatives, R₁ and R₂ are hydrogen and R₃ is hydrogen or methyl. Depending on the choice of R₃, it is an acrylate (hydrogen) or methacrylate (methyl).

The C₁₋₁₈ alkyl groups that are optionally substituted by —OH or —SO₃M and that are possible for X are preferably selected from methyl, ethyl, C₂₋₄ hydroxyalkyl, C₂₋₄sulfoalkyl, and C₄₋₁₈ alkyl groups.

The C₂₋₄ hydroxyalkyl groups may be selected from ethyl, n-propyl, isopropyl, and n-butyl. The n-butyl, sec-butyl, tert-butyl, pentyl, hexyl, ethylhexyl, octyl, decyl, dodecyl, or stearyl groups can be mentioned as examples of unsubstituted C₄₋₁₈ alkyl groups. Of these, n-butyl and ethylhexyl are particularly suitable. Ethylhexyl is, in particular, 2-ethylhexyl. 2-sulfoethyl and 3-sulfopropyl, in particular, can be mentioned as C₂₋₄ sulfoalkyl groups.

In one embodiment of the acrylic acid derivatives, R₄ is —NR₅R₆, R₅ being H and R₆ being 2-methyl-propan-2-yl-1-sulfonic acid. In particular, R₁, R₂, and R₃ are hydrogen.

According to one embodiment, R₄ is —OX and X is hydrogen. In particular, R₁, R₂, and R₅ are hydrogen (acrylic acid). Alternatively, R₃ is methyl (methacrylate). In one embodiment of the acrylic acid derivatives, R₄ is —OX and X is methyl. In particular, R₁, R₂, and R₅ are hydrogen (methyl acrylate). According to one embodiment, R₄ is —OX and X is 2-ethylhexyl. In particular, R₁, R₂, and R₃ are hydrogen (ethyl acetate). According to one embodiment, R₄ is —OX and X is n-butyl. In particular, R₁, R₂, and R₅ are hydrogen (n-butyl acrylate). In one embodiment of the acrylic acid derivatives, R₄ is —OX and X is 2-sulfoethyl. In particular, R₁, R₂, and R₃ are hydrogen (sulfoethyl acrylate). In one embodiment of the acrylic acid derivatives, R₄ is —OX and X is 3-sulfopropyl. In particular, R₁ and R₂ are hydrogen and R₃ is methyl (sulfopropyl (meth)acrylate).

In various embodiments, R₄ represents —OX, X being hydrogen, C₁₋₁₀ alkyl, an alkali metal, or an ammonium group, preferably C₁₋₁₀ alkyl, more preferably methyl, n-butyl, or ethylhexyl, in particular 2-ethylhexyl.

The polymers or copolymers constructed from monomers of the formula (I) typically satisfy the formula (II):

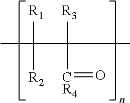

R₁-R₄ having the above-mentioned meanings and n being an integer of at least 3. n may be, for example, greater than 5, 10, 20, 30, 40, 50, 60, 70, 80, or 100. n may be, for example, less than 10,000, 7,500, 5,000, 2,500, 1,000, or 500. According to one embodiment, n is in the range of from 5 to 5,000. In one embodiment, n is in the range of from 10 to 1,000.

The group of these polymers and copolymers represents a meaningful generalization of the copolymers present in Dimension PA 140. The emulsion stabilizer is preferably an acrylate copolymer which comprises at least two different monomers of the formula (I). In various embodiments, the copolymer contains AMPS, optionally in combination with (meth)acrylic acid and/or at least one alkyl (meth)acrylate. According to one embodiment, the copolymer contains

AMPS and one or more monomers selected from acrylate, methacrylate, methyl acrylate, ethylhexyl acrylate, n-butyl acrylate, n-vinylpyrrolidone, and styrene.

According to one embodiment, the copolymer contains AMPS, acrylate, methyl acrylate, and styrene. According to one embodiment, the copolymer contains AMPS, acrylate, methyl acrylate, and ethyl hexacrylate. According to one embodiment, the copolymer contains AMPS, methyl acrylate, n-vinylpyrrolidone, and styrene. According to one embodiment, the copolymer contains AMPS, acrylate, methyl acrylate, and ethyl hexacrylate. According to one embodiment, the copolymer contains AMPS, methyl acrylate, n-vinylpyrrolidone, and styrene. According to one embodiment, the copolymer contains AMPS, methyl acrylate, and styrene. According to one embodiment, the copolymer contains AMPS, methacrylate, and styrene. According to one embodiment, the copolymer contains AMPS, acrylate, methyl acrylate, and n-butyl acrylate.

According to one embodiment, the emulsion stabilizer is a copolymer as defined in EP0562344B1, which is incorporated by reference herein. According to one embodiment, the emulsion stabilizer is a copolymer containing a) AMPS, sulfoethyl or sulfopropyl(meth)acrylate, or vinylsulfonic acid, in particular in a proportion of 20 to 90%; b) a vinylically unsaturated acid, in particular in a proportion of 0 to 50%; c) methyl or ethyl acrylate or methacrylate, C₂₋₄ hydroxyalkyl acrylate, or N-vinylpyrrolidone, in particular in a proportion of 0 to 70%; and d) styrene or C₄₋₁₈ alkyl acrylate or C418 alkyl methacrylate, in particular in a proportion of 0.1 to 10%.

According to one embodiment, the emulsion stabilizer is a copolymer containing 2-acrylamido-2-methylpropane sulfonic acid, sulfoethyl or sulfopropyl (meth)acrylate, or vinylsulfonic acid, in particular in a proportion of 40 to 75%; b) acrylic acid or methacrylic acid, in particular in a proportion of 10 to 40%; c) methyl or ethyl acrylate or methacrylate, C₂₋₄ hydroxyalkyl acrylate, or N-vinylpyrrolidone, in particular in a proportion of 10 to 50%; and d) 0.5 to 5% styrene or C₄₋₁₈ alkyl acrylate or methacrylate, in particular in a proportion of 0.5 to 5%.

According to one embodiment, the emulsion stabilizer is a copolymer containing a) 40 to 75% 2-acrylamido-2-methylpropane sulfonic acid, sulfoethyl or sulfopropyl (meth)acrylate, or vinylsulfonic acid, in particular in a proportion of 40 to 75%; b) acrylic acid or methacrylic acid, 10 to 30%; c) methyl or ethyl acrylate or methacrylate, or N-vinylpyrrolidone, in particular in a proportion of 10 to 50%; and d) styrene or C₄₋₁₈ alkyl acrylate or methacrylate, in particular in a proportion of 0.5 to 5%.

A suitable copolymer is available, for example, under the trade name Dimension PA 140 (Solenis).

According to one embodiment, the emulsion stabilizer does not consist of or comprise N-vinylpyrrolidone, polyvinylpyrrolidine homopolymer, or polyvinylpyrrolidine copolymer.

The exact determination of the proportion of emulsion stabilizer in the stabilization layer is technically difficult. However, in contrast to the use as a protective colloid during the encapsulation of the core material, it is assumed that a substantial part of the emulsion stabilizer is integrated in the microcapsule shell.

The proportion of the emulsion stabilizer used in the components used for the microencapsulation may be in the range from 0.1 to 15 wt. %. For example, the proportion of the emulsion stabilizer used may be 0.1 wt. %, 0.2 wt. %, 0.5 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. Below a concentration of 0.1 wt. %, there is the risk that the surface of the barrier layer is not sufficiently covered with the emulsion stabilizer in order to ensure the effect according to the invention, namely the increase in the amount of the stability layer deposited. Above 15 wt. %, the high concentration of an emulsion stabilizer can hinder the formation of the stability layer. In a preferred embodiment, the emulsion stabilizer is used in a proportion in the components used for the microencapsulation of from 0.25 wt. % to 5 wt. %. In a particularly preferred embodiment, the proportion of the emulsion stabilizer used is in the range of from 0.5 wt. % to 4 wt. %.

Based on the assumption that at least some of the emulsion stabilizer is incorporated in the microcapsule wall, according to one embodiment the proportion of the emulsion stabilizer is in the range of from 0.5 to 15.0 wt. %, based on the total weight of the microcapsule wall. For example, the proportion of the emulsion stabilizer used may be 0.5 wt. %, 1.0 wt. %, 1.5 wt. %, 2.0 wt. %, 2.5 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. In a preferred embodiment, the proportion of the wall-forming components of the microcapsule shell is in the range of from 1 wt. % to 11 wt. %. In a particularly preferred embodiment, the proportion of the emulsion stabilizer used is in the range of from 2 wt. % to 7 wt. %.

The barrier layer preferably contains, as a wall former, one or more components selected from the group consisting of an aldehyde component, an aromatic alcohol, an amine component, and an acrylate component. Preparation methods for preparing microcapsules having these wall materials are known to a person skilled in the art. A polymer selected from a polycondensation product of an aldehyde component comprising one or more aromatic alcohols and/or amine components can be used to produce the barrier layer.

As shown in embodiments 2 and 3, the thin wall thickness of the barrier layer can be achieved in particular with a melamine-formaldehyde layer containing aromatic alcohols or m- aminophenol. Consequently, the barrier layer preferably comprises an aldehyde component, an amine component, and an aromatic alcohol.

The use of amine-aldehyde compounds in the barrier layer, in particular melamine formaldehyde, has the advantage that these compounds form a hydrophilic surface with a high proportion of hydroxyl functionality, which thus exhibit a fundamental compatibility with the hydrogen bond-oriented components of the first layer (stability layer), such as biodegradable proteins, polysaccharides, chitosan, lignins, and phosphazenes, but also inorganic wall materials such as CaCO₃ and polysiloxanes. Equally, polyacrylates, in particular from the components styrene, vinyl compounds, methyl methacrylate, and 1,4-butanediol acrylate, methacrylic acid, can equally be produced as the microcapsule wall by means of initiation, for example, with t-butyl hydroperoxide in a free-radical induced polymerization (polyacrylates), which polyacrylates form a hydrophilic surface having a high proportion of hydroxyl functionality and which are therefore equally compatible with the components according to the invention of the stability layer.

In a preferred embodiment, a wall former of the barrier layer is thus an aldehyde component. According to one embodiment, the aldehyde component of the barrier layer is selected from the group consisting of formaldehyde, glutaraldehyde, succinaldehyde, furfural, and glyoxal. All these aldehydes have successfully been used to produce microcapsules (see WO 2013 037 575 A1), and therefore it can be assumed that similarly dense capsules can be obtained with them as with formaldehyde.

Based on the examples, for the formation of the wall, the proportion of the aldehyde component should be in the range of from 5 wt. % to 50 wt. % based on the total weight of the barrier layer. For example, the proportion of the aldehyde component may be 5 wt. %, 6 wt. %, 7 wt. %, 8 wt. %, 9 wt. %, 10 wt. %, or 15 wt. %, 20 wt. %, 25 wt. %, 30 wt. %, 35 wt. %, 40 wt. %, 45 wt. %, or 50 wt. %. It is assumed that, outside these limits, a sufficiently stable and dense, thin layer cannot be obtained. The concentration of the aldehyde component in the barrier layer is preferably in the range of from 10 wt. % to 30 wt. %. Particularly preferably, the concentration of the aldehyde component in the barrier layer is in the range of from 15 wt. % to 20 wt. %.

Suitable amine components in the barrier layer are, in particular, melamine, melamine derivatives, and urea or combinations thereof. Suitable melamine derivatives are etherified melamine derivatives and also methylolated melamine derivatives. Melamine in the methylolated form is preferred. The amine components may be used, for example, in the form of alkylated mono- and polymethylol urea precondensation products or partially methylolated mono- and polymethylol-1,3,5-triamono-2,4,6-triazine precondensation products such as Dimension SD® (from Solenis). According to one embodiment, the amine component is melamine. According to an alternative embodiment, the amine component is a combination of melamine and urea.

The aldehyde component and the amine component may be present in a molar ratio ranging from 1:5 to 3:1. For example, the molar ratio may be 1:5, 1:4.5, 1:4, 1:3.5, 1:3, 1:2.5, 1:2, 1:1.8, 1:1.6, 1:1.4, 1:1.35, 1;1.3, 1:1.2, 1:1, 1.5:1, 2:1, 2.5:1, or 3:1. The molar ratio is preferably in the range of from 1:3 to 2:1. Particularly preferably, the molar ratio of the aldehyde component and the amine component may be in the range of from 1:2 to 1:1. The aldehyde component and the amine component are generally used in a ratio of about 1:1.35. This molar ratio allows for complete reaction of the two reactants and leads to a high degree of tightness of the capsules. For example, aldehyde-amine capsule walls are also known with a molar ratio of 1:2. These capsules have the advantage that the proportion of the high-crosslinking aldehyde, in particular formaldehyde, is very low. However, these capsules have a lower tightness than the capsules with a ratio of 1:1.35. Capsules with a ratio of 2:1 have increased tightness, but have the disadvantage that the aldehyde component is present in partially unreacted form in the capsule wall and in the slurry.

In one embodiment, the proportion of the amine component(s) (for example melamine and/or urea) in the barrier layer is in the range of from 20 wt. % to 85 wt. %, based on the total weight of the barrier layer. For example, the proportion of the amine component may be 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, or 85 wt. %. In a preferred embodiment, the proportion of the amine component in the barrier layer with respect to the total weight of the barrier layer is in the range of from 40 wt. % to 80 wt. %. The proportion of the amine component is particularly preferably in the range of from 55 to 70 wt. %.

By virtue of the aromatic alcohol, it is possible to greatly reduce the wall thickness of the barrier layer composed of the amine component and the aldehyde component, while also obtaining a layer which has the necessary tightness and which is stable enough at least in combination with the stability layer. The aromatic alcohols give the wall increased tightness, since their highly hydrophobic aromatic structure makes it difficult for low-molecular substances to diffuse through. As shown in the examples, phloroglucinol, resorcinol, or m-aminophenol are particularly suitable as the aromatic alcohol. Consequently, in one embodiment, the aromatic alcohol is selected from the group consisting of phloroglucinol, resorcinol, and aminophenol. In combination with the amine and aldehyde component, the aromatic alcohol is used in a molar ratio to the aldehyde component in the range of from (alcohol:aldehyde) 1:1 to 1:20, preferably in the range of from 1:2 to 1:10.

In one embodiment, the proportion of the aromatic alcohol in the barrier layer relative to the total weight of the barrier layer is in the range of from 1.0 wt. % to 20 wt. %. For example, the proportion of the aromatic alcohol may be 1.5 wt. %, 2.0 wt. %, 2.5 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, or 20 wt. %. Owing to their aromatic structure, the aromatic alcohols give the capsule wall a coloring which increases with the proportion of the aromatic alcohol. Such a coloring is undesirable in many applications. In addition, the aromatic alcohols are susceptible to oxidation, which leads to a change in the coloring over time. As a result, the undesired coloration of the microcapsules can be poorly compensated with a dye. Therefore, the aromatic alcohols should not be used above 20.0 wt. %. Below 1.0 wt. %, no effect is detectable with respect to the tightness. In a preferred embodiment, the proportion of the aromatic alcohol in the barrier layer relative to the total weight of the barrier layer is in the range of from 5.0 wt. % to 15.0 wt. %. Up to a percentage of 15.0 wt. %, coloration is tolerable in most applications. In a particularly preferred embodiment, the proportion of the aromatic alcohol in the barrier layer with respect to the total weight of the barrier layer is in the range of from 6 wt. % to 16.0 wt. %. In particular, the proportion of the aromatic alcohol in the barrier layer is in the range of from 10 wt. % to 14.0 wt. %.

In another embodiment, the aldehyde component of the barrier layer can be used together with an aromatic alcohol such as resorcinol, phloroglucinol, or m-aminophenol as the wall-forming component(s), i.e. without the amine component(s).

In one embodiment, the barrier layer comprises melamine, formaldehyde, and resorcinol. In one embodiment, the barrier layer of the microcapsules comprises melamine, urea, formaldehyde, and resorcinol. In a preferred embodiment, the barrier layer contains melamine in the range of from 25 to 40 wt. %, formaldehyde in the range of from 15 to 20 wt. %, and resorcinol in the range of from 10 to 14 wt. %, and optionally urea in the range of from 25 to 35 wt. %. The proportions refer to the amounts used for the formation of the wall of the layer and are based on the total weight of the barrier layer without a protective colloid.

To encapsulate the core material with the barrier layer consisting of an aldehyde component, an amine component, and an aromatic alcohol, an emulsion stabilizer is preferably used as the protective colloid, as mentioned above. The emulsion stabilizer used as a protective colloid may be a polymer or copolymer as defined above as a mediator agent. For example, the protective colloid is a copolymer AMPS (Dimension® PA 140, Solenis) or the salts thereof. In one embodiment, the same copolymer is used as the protective colloid and as the mediator agent.

Suitable amine components in the barrier layer are, in particular, melamine, melamine derivatives, and urea or combinations thereof. Suitable melamine derivatives are etherified melamine derivatives and also methylolated melamine derivatives. Melamine in the methylolated form is preferred. The amine components may be used, for example, in the form of alkylated mono- and polymethylol urea precondensation products or partially methylolated mono- and polymethylol-1,3,5-triamono-2,4,6-triazine precondensation products such as Dimension SD® (from Solenis). According to one embodiment, the amine component is melamine. According to an alternative embodiment, the amine component is a combination of melamine and urea.

The stability layer forms the majority of the microcapsule shell and thus ensures a high biodegradability of at least 40% within 60 days according to OECD 301 F. Biopolymers suitable for the stability layer are proteins such as gelatin, whey protein, plant storage protein; polysaccharides such as alginate, gum arabic, modified gum, chitin, dextran, dextrin, pectin, cellulose, modified cellulose, hemicellulose, starch, or modified starch; phenolic macromolecules such as lignin; polyglucosamines such as chitosan; polyvinyl esters such as polyvinyl alcohols and polyvinyl acetate; phosphazenes and polyesters such as polylactide or polyhydroxyalkanoate. This enumeration of the specific components in the individual substance classes is merely exemplary and is not to be understood as limiting. Suitable natural wall formers are known to a person skilled in the art. Furthermore, the various methods for wall formation, for example coacervation or interfacial polymerization, are known to a person skilled in the art.

The biopolymers can be selected accordingly for the relevant application in order to form a stable multilayer shell with the material of the stability layer. In addition, the biopolymers can be selected to achieve compatibility with the chemical conditions of the field of application. The biopolymers can be combined as desired in order to influence the biodegradability or, for example, the stability and chemical resistance of the microcapsule.

In one embodiment of the first aspect, the shell of the microcapsules has a biodegradability of 50% according to OECD 301 F. In another embodiment, the shell of the microcapsule has a biodegradability of at least 60% (OECD 301 F). In another embodiment, the biodegradability is at least 70% (OECD 301 F). Biodegradability is measured in each case over a period of 60 days. In the extended degradation method (“enhanced ready biodegradation”), the biodegradability is measured over a period of 60 days (see Opinion on an Annex XV dossier proposing restrictions on intentionally-added microplastics of Jun. 11, 2020 ECHA/RAC/RES-O-0000006790-71-01/F)). Preferably, the microcapsules are freed from residues by means of washing prior to the determination of the biodegradability. Particularly preferably, copies of the microcapsules are produced for this test with an inert, non-biodegradable core material such as perfluorooctane (PFO) instead of the perfume oil. In one embodiment, after being prepared, the capsule dispersion is washed by centrifugation and redispersion in distilled water three times. . For this purpose, the sample is centrifuged (e.g. for 10 min at 12,000 RPM). After the clear supernatant has been vacuumed off, water is added and the sediment is redispersed by means of shaking. In the measurement of biodegradability, various reference samples can be used, such as the rapidly degradable ethylene glycol or natural-based walnut shell flour with the typical step-like degradation of a complex substance mixture. The microcapsule according to the invention shows a similar, preferably better, biodegradability over a period of 28 or 60 days than the walnut shell flour.

Residues in the microcapsule dispersions are substances which are used in the preparation of the microcapsules and are in non-covalent interaction with the shell, such as deposition aids, preservatives, emulsifiers/protective colloids, or excess feedstocks. These residues have a proven influence on the biodegradability of microcapsule dispersions. For this reason, washing is necessary before determining the biodegradability.

In order to get an impression of the proportion of covalently bonded and non-covalently bonded constituents in the microcapsule dispersion, the capsules were investigated by means of the quantification method described in Gasparini et al. 2020 on the basis of Py-GC-MS for polymer-encapsulated fragrances. This method includes a multistage purification protocol for polymers of complex samples such as microcapsule dispersions, and makes it possible to quantify volatile residual constituents suspected of not being covalently incorporated into the 3D polymer network and therefore not being quantifiable by other standard methods (e.g. SPME-GC-MS or TGA). Based on this method, it was confirmed that individual layers of the microcapsule according to the invention, in particular the barrier layer and the stability layer, can be inseparably connected and regarded as a monopolymer. It can be assumed that, by adding the emulsion stabilizer, not only the structural accommodation of the stability layer by the barrier layer is improved, but additionally the structural (covalent) bonding of all wall-forming components is increased.

A high level of biodegradability according to the invention is achieved on the one hand by the wall formers used and on the other hand by the structure of the shell according to the invention. This is because the use of a particular percentage of biopolymers does not automatically lead to a corresponding value of the biodegradability. This is dependent on how the biopolymers are present in the shell.

According to a preferred embodiment, the stability layer contains gelatin as the biopolymer. According to another preferred embodiment, the stability layer contains alginate as the biopolymer. According to another preferred embodiment, the stability layer contains gelatin and alginate as biopolymers. As shown in the embodiment, both gelatin and alginate are suitable for the preparation of microcapsules according to the invention with high biodegradability and high stability. In particular it was able to be demonstrated that, in the case of a stability layer containing gelatin and alginate, the treatment of the surface of the barrier layer with an emulsion stabilizer, in particular an AMPS-containing copolymer, can lead to a significant increase in the layer thickness of the stability layer (see Examples 1-4). Further suitable combinations of natural components in the first layer (stability layer) are gelatin and gum arabic.

The stability layer contains one or more curing agents. Curing agents according to the invention are aldehydes such as glutaraldehyde, formaldehyde, glyoxal, tannins, enzymes such as transglutaminase, organic anhydrides such as maleic anhydride, epoxy compounds, polyvalent metal cations, amines, polyphenols, maleimides, sulfides, phenol oxides, hydrazides, isocyanates, isothiocyanates, N-hydroxysulfosuccinimide derivatives, carbodiimide derivatives, and polyols.

Preferably the curing agent is glutaraldehyde due to its very good crosslinking properties. The curing agent glyoxal is also preferred due to its good crosslinking properties and, compared with glutaraldehyde, lower toxicological classification. The use of curing agents produces a higher tightness of the stability layer. However, curing agents lead to reduced biodegradability of the natural polymers.

According to one embodiment, the barrier layers do not contain isocyanates. Some isocyanates such as methylene diphenyl isocyanate (MDI), hexamethylene diisocyanate (HDI) or toluene-2,4-diisocyanate (TDI) have a certain toxicity and are to be assessed critically from the viewpoint of occupational safety. Furthermore, side reactions with components of the core material can also occur in isocyanates.

According to one embodiment, the barrier layers according to the invention contain no silane monomers, silane oligomers, or silicates. These components can be disadvantageous in certain combinations for the formation of the capsule according to the invention. For example, it is known to a person skilled in the art that silicates such as TEOS and TMOS (tetraethyl orthosilicate or tetramethyl orthosilicate) undergo side reactions with their constituents, for example fragrances, when added to an oil phase and thus influence the properties of the oil phase, i.e., the core material (e.g., the fragrance oil), for example negatively.

Furthermore, the TEOS and TMOS are classified as critical for reasons of occupational safety due to ease of inflammation and toxicity and are preferably not used according to the invention.

According to one embodiment, the barrier layers do not contain a silicone-melamine polyurethane copolymer. A silicone-melamine polyurethane copolymer can also result in side reactions with the core material, i.e., the oil phase, in particular fragrances located therein. Furthermore, a silicone-melamine polyurethane copolymer is also classified as critical with respect to occupational safety.

Due to the presence of the barrier layer as a diffusion barrier, the amount of curing agent in the stability layer can be kept low, which in turn contributes to the easy biodegradability of the layer. According to one embodiment, the proportion of the curing agent in the stability layer is below 25 wt. %. Unless explicitly defined otherwise, the proportions of the constituents of the layers are based on the total weight of the layer, i.e. the total dry weight of the constituents used for preparation, without taking into account the constituents used during preparation, which are not incorporated or only slightly incorporated into the layer, for example surfactants and protective colloids. Above this value, the biodegradability according to the invention according to OECD 301 F cannot be guaranteed. The proportion of the curing agent in the stability layer may be, for example, 1.0 wt. %, 2.0 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, or 24 wt. %. The proportion of the curing agent in the stability layer is preferably in the range of from 1 to 15 wt. %. This proportion leads to effective crosslinking of the gelatin and, in a quantitative reaction, results in as little residual monomer as possible being formed. The range 9 to 12 wt. % is particularly preferred, since it ensures the required degree of crosslinking as well as a stable envelope for the barrier layer in order to buffer the otherwise sensitive barrier layer, and has only little residual aldehyde, which is degraded in a downstream alkaline setting of the slurry by means of an aldol reaction.

In one embodiment, the stability layer contains gelatin and glutaraldehyde. According to another embodiment, the stability layer contains gelatin, alginate, and glutaraldehyde. In an additional embodiment, the stability layer contains gelatin and glyoxal. According to another embodiment, the stability layer contains gelatin, alginate, and glyoxal. The exact chemical composition of the stability layer is not crucial. However, the desired effect is preferably achieved with polar biopolymers.

The use of the emulsion stabilizer according to the invention on the surface of the barrier layer significantly increases the mean thickness of the stability layer. The mean thickness of the stability layer is at least 1 μm. The mean thickness of the stability layer may be 1 μm, 1.2 μm, 1.4 μm, 1.6 μm, 1.8 μm, 2 μm, 2.2 μm, 2.4 μm, 2.6 μm, 2.8 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. The stability layer frequently has an elliptical shape in cross section, and therefore the thickness of the stability layer varies over the microcapsule surface. Therefore, a mean thickness of the microcapsules is calculated. In addition, the deposition varies from microcapsule to microcapsule. This is taken into account in that the mean thickness of several microcapsules is determined and the average is calculated therefrom. Thus, the mean thickness mentioned here is, strictly speaking, an average mean thickness. The layer thickness of the stability layer can be determined in two ways according to the invention. Firstly, there is the light microscopic approach, i.e. the direct optical measurement of the observed layer thickness by means of a microscope and corresponding software. In this case, a large number of microcapsules of a dispersion are measured and at least the diameter of each individual microcapsule is determined based on the variation within the capsules.

A second possibility is the measurement of the particle size distribution by means of laser diffraction. Here, the modal value of a particle size distribution of the without the layer to be measured can be compared with the modal value of a particle size distribution with the layer to be measured. The increase in this modal value reflects the increase in the hydrodynamic diameter of the major fraction on measured microcapsules. The formation of the difference from the two measured modal values ultimately results in twice the layer thickness of the layer.

According to a preferred embodiment, the mean thickness of the stability layer is at least 2 μm. By choosing a suitable combination of emulsion stabilizer and wall former for the stability layer, stability layers having a mean thickness of 6 μm or more can be formed. In a particularly preferred embodiment, the mean thickness of the stability layer is at least 3 μm.

In contrast to other biodegradable microcapsules, the microcapsules according to the invention are very tight. According to one embodiment, the microcapsules have a tightness which ensures egress of at most 50 wt. % of the core material used after storage over a period of 4 weeks at a temperature of 0 to 40° C.

In various embodiments, the microcapsules according to the invention have a tightness which is at most 80 wt. % of the core material used after storage for a period of 12 weeks at a temperature of 0 to 40° C., preferably at most 75 wt. % and particularly preferably at most 70 wt. %. In various embodiments, the microcapsules according to the invention contain, after storage for a period of 12 weeks at a temperature of 0 to 40° C., thus still at least 20 wt. %, preferably at least 25 wt. %, and in particular at least 30 wt. % of the core material used.

In further embodiments, the microcapsules according to the invention still contain at least 50 wt. % of the core material used after storage over a period of 4 weeks at a temperature of 0 to 40° C.

In addition to the shell material, the tightness also depends on the type of core material. The tightness of the microcapsules according to the invention was determined according to the invention for the fragrance oil Weiroclean from Kitzing, since the chemical properties of this fragrance oil are representative of microencapsulated fragrance oils. Weiroclean has the following components (with proportion based on the total weight):

| | |
|---|---|
| 1-(1,2,3,4,5,6,7,8-Octahydro-2,3,8,8-tetramethyl-2-naphthalenyl)ethanone | 25-50% |
| Benzoic acid, 2-hydroxy, 2-hexyl ester | 10-25% |
| Phenylmethyl benzoate | 5-10% |
| 3-Methyl-4-(2,6,6-trimethyl-2-cyclohexenyl)-3-buten-2-one | 1-5% |
| 3,7-Dimethyl-6-octen-1-ol | 1-5% |
| 3-methyl-5-phenylimidazoles | 1-5% |
| 2,6-Dimethyloct-7-en-2-ol | 1-5% |
| 4-(2,6,6-Trimethylcyclohex-1-enyl)-but-3-en-2-one | 1-5% |
| 3a,4,5,6,7,7a-Hexahydro-4,7-methano-1H-indene-6-yl-propanoate | 1-5% |
| 2-tert-Butylcyclohexyl acetate | 1-5% |
| 2-Heptyl cyclopentanone | 1-5% |
| Pentadecan-15-olide | 1-5% |
| 2H-1-Benzopyran-2-one | 0.1-1% |
| 2,6-Di-tert-butyl-p-cresol | 0.1-1% |
| 4-Methyl-3-decen-5-ol | 0.1-1% |
| 2,4-Dimethyl-3-cyclohexene-1-carboxaldehyde | 0.1-1% |
| [(2E)-3,7-Dimethylocta-2,6-dienyl] acetate | 0.1-1% |
| Allyl hexanoate | 0.1-1% |
| 2-Methylundecanal | 0.1-1% |
| 10-Undecenal | 0.1-1% |
| cis-3,7-Dimethyl-2,6-octadienyl ethanoate | 0.1-1% |
| 3,7,11-Trimethyldodeca-1,6,10-trien-3-ol | 0.1-1% |
| Undecan-2-one | 0.1-1% |

A large number of different materials are suitable as the core material, inter alia fragrances and cosmetic active ingredients. According to a preferred embodiment of the microcapsules according to the invention, the core material is hydrophobic. The core material may be solid or liquid. In particular, it is liquid. It is preferably a liquid hydrophobic core material. In a preferred embodiment, the core material is a fragrance or the core material comprises at least one fragrance. Particularly preferably, the core material consists of fragrance oils or perfume oils that are optimized for microencapsulation for the field of detergents and cleaning agents, for example the fragrance formulation Weiroclean (from Kurt Kitzing GmbH). The fragrances can be used in the form of a solid or liquid formulation, but in particular in liquid form.

Fragrances that can be used as the core material are not particularly limited. Thus, individual fragrance compounds of natural or synthetic origin, for example of the ester, ether, aldehyde, ketone, alcohol and hydrocarbon type, can be used. Fragrance compounds of the ester type are e.g., benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmacyclate. The ethers include, for example, benzyl ethyl ether and ambroxan, the aldehydes include the ones mentioned above, for example the linear alkanals having 8 to 18 carbon atoms, citral, citronellal, citronellyloxyacetaldehyde, cyclamenaldehyde (3-(4-propan-2-ylphenyl)butanal), lilial and bourgeonal, the ketones include, for example, the ionones, [alpha]-isomethylionone and methylcedrylketone, the alcohols include anethole, citronellol, eugenol, geraniol, linalool, phenylethyl alcohol and terpineol, the hydrocarbons mainly include terpenes such as limonene and pinene. Preferably, however, mixtures of different fragrances are used which together produce an appealing scent.

Suitable aromatic aldehydes may be selected from adoxal (2,6,10-trimethyl-9-undecenal), anisaldehyde (4-methoxybenzaldehyde), cymal or cyclamen aldehyde (3-(4-isopropylphenyl)-2-methylpropanal), nympheal (3-(4-isobutyl-2-methylphenyl)propanal), ethyl vanillin, florhydral (3-(3-isopropylphenyl)butanal]), trifernal (3-phenylbutyraldehyde), helional (3-(3,4-methylenedioxyphenyl)-2-methylpropanal), heliotropin, hydroxycitronellal, lauraldehyde, lyral (3- and 4-(4-hydroxy-4-methylpentyl)-3-cyclohexene-1-carboxaldehyde), methyl nonyl acetaldehyde, lilial (3-(4-tert-butylphenyl)-2-methylpropanal), phenyl acetaldehyde, undecylenic aldehyde, vanillin, 2,6, 10-trimethyl-9-undecenal, 3-dodecen-1-al, alpha-n-amyl cinnamic aldehyde, melonal (2,6-dimethyl-5-heptenal), triplal (2,4-dimethyl-3-cyclohexene-1-carboxaldehyde), 4-methoxybenzaldehyde, benzaldehyde, 3-(4-tert-butylphenyl)propanal, 2-methyl-3-(paramethoxyphenyl)propanal, 2-methyl-4-(2,6,6-timethyl-2(1)-cyclohexene-1- yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4- isopropylbenzylaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methano-1H-indene carboxaldehyde, 3-ethoxy-4-hydroxybenzaldehyde, para-ethyl-alpha, alpha-dimethyl hydrocinnamic aldehyde, alpha-methyl-3,4-(methylenedioxy)-hydrocinnamic aldehyde, 3,4-methylenedioxybenzaldehyde, alpha-n-hexyl cinnamic aldehyde, m-cymene-7-carboxaldehyde, alpha-methyl phenyl acetaldehyde, tetrahydrocitral (3,7-dimethyloctanal), undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethylcyclohexene-3-carboxaldehyde, 4-(4-hydroxy-4-methylpentyl)-3-cylohexene-1-carboxaldehyde, 7-methoxy-3,7-dimethyloctan-1-al, 2-methyldecanal, 1-nonanal, 1-octanal, 2,6, 10-trimethyl-5,9-undecadienal, 2-methyl-3-(4-tert-butyl)propanal, dihydrocinnamic aldehyde, 1-methyl-4-(4-methyl-3-pentenyl)-3-cyclohexene-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexene carboxaldehyde, 7-hydroxy-3,7-dimethyloctanal, trans-4-decenal, 2,6-nonadienal, para-tolyl acetaldehyde, 4-methylphenylacetaldehyde, 2-methyl-4-(2,6,6-trimethyl-1-cyclohexen-1-yl)-2-butenal, ortho-methoxy cinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzene acetaldehyde, 6,6-dimethyl-2-norpinen-2-propionaldehyde, para-methyl phenoxy acetaldehyde, 2-methyl-3-phenyl-2-propen-1-al, 3,5,5-trimethylhexanal, hexahydro-8,8-dimethyl-2-naphthaldehyde, 3-propylbicyclo[2.2.1]-hept-5-ene-2-carbaldehyde, 9-decenal, 3-methyl-5-phenyl-1-pentanal, floral (4,8-dimethyl-4,9-decadienal), aldehyde C12MNA (2-methylundecanal), liminal (beta-4-dimethylcyclohex-3-ene-1-propan-1-al), methyl nonyl acetaldehyde, hexanal, trans-2-hexenal, and mixtures thereof.

Suitable aromatic ketones include, but are not limited to, methyl beta-naphthyl ketone, musk indanone (1,2,3,5,6,7-hexahydro-1,1,2,3,3-pentamethyl-4H-inden-4-one), calone (methylbenzodioxepinone), tonalid (6-acetyl-1,1,2,4,4,7-hexamethyltetraline), alpha-damascone, beta-damascone, delta-damascone, iso-damascone, damascenone, methyl dihydrojasmonate (hedione), menthone, carvone, camphor, koavone (3,4,5,6,6-pentamethylhept-3-en-2-one), fenchone, alpha-ionone, beta-ionone, dihydro-beta-ionone, gamma-methyl ionone, fleuramone (2-heptylcyclopentanone), frambinone methyl ether (4-(4-methoxyphenyl)butan-2-one), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl beta-naphthyl ketone, benzylacetone, benzophenone, para-6- hydroxyphenyl butanone, celery ketone (3-methyl-5-propyl-2-cyclohexenone), isopropyldecahydro-2-naphtone, dimethyl octenone, frescomenthe (2-butan-2-ylcyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)indanone, 4-damascol, dulcinyl (4-(1,3-benzodioxol-5-yl)butan-2-one), hexalone (1-(2,6,6-trimethyl-2-cyclohexen-1-yl)-1,6-heptadien-3-one), isocyclemone E (2-acetonaphthone-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butylcyclohexanone, delphone (2-pentylcyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one), 2,4,4,7-tetramethyloct-6-en-3-one, tetrameran (6,10-dimethylundecen-2-one), and mixtures thereof.

The core materials may also contain natural fragrance mixtures, such as those that can be are obtained from plant sources, for example pine, citrus, jasmine, patchouli, rose, or ylang-ylang oil. Equally, clary sage oil, chamomile oil, clove oil, melissa oil, mint oil, cinnamon leaf oil, lime blossom oil, juniper berry oil, vetiver oil, olibanum oil, galbanum oil, and labdanum oil, as well as orange blossom oil, neroli oil, orange peel oil, and sandalwood oil are also suitable. Further conventional fragrances which can be contained in the agents according to the invention in the context of the present invention are, for example, the essential oils such as angelica root oil, anise oil, arnica flower oil, basil oil, bay oil, champak flower oil, noble fir oil, noble fir cone oil, elemi oil, eucalyptus oil, fennel oil, spruce needle oil, galbanum oil, geranium oil, ginger grass oil, guaiacum wood oil, gurjun balsam oil, helichrysum oil, ho oil, ginger oil, iris oil, cajeput oil, calamus oil, chamomile oil, camphor oil, canaga oil, cardamom oil, cassia oil, pine needle oil, copaiva balsam oil, coriander oil, spearmint oil, caraway oil, cumin oil, lavender oil, lemon grass oil, lime oil, mandarin oil, lemon balm oil, musk seed oil, myrrh oil, clove oil, neroli oil, niaouli oil, olibanum oil, oregano oil, palmarosa oil, patchouli oil, Peru balsam oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil as well as ambrettolide, ambroxan, a-amyl cinnammaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornylacetate, boisambrene forte, α-bromostyrene, n-decylaldehyde, n-dodecyl aldehyde, eugenol, eugenol methyl ether, eucalyptol, farnesol, fenchone, fenchyl acetate, geranyl acetate, geranyl formate, heliotropin, heptin carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvactol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl-β naphthyl ketone, methyl-n-nonyl acetaldehyde, methyl-n-nonyl ketone, muscone, B-naphthol ethyl ether, β-naphthol methyl ether, nerol, n-nonyl aldehyde, nonyl alcohol, n-octyl aldehyde, p-oxy-acetophenone, pentadecanolide, β-phenylethyl alcohol, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undecalactone, vanillin, veratraldehyde, cinnamaldehyde, cinnamyl alcohol, cinnamic acid, cinnamic acid ethyl ester, cinnamic acid benzyl ester, diphenyl oxide, limonene, linalool, linalyl acetate and propionate, melusate, menthol, menthone, methyl-n-heptenone, pinene, phenylacetaldehyde, terpinyl acetate, citral, citronellal, and mixtures thereof.

### Perfume Composition

In various embodiments, at least one perfume composition is used as core material and is, in particular, liquid. The perfume composition encapsulated in the described microcapsules preferably comprises, based on the total weight of all fragrances contained in the perfume composition:
a) ≤10 wt. %, preferably ≤8 wt. %, of odorants having a CLogP of ≤2.5 and a boiling point of ≥200° C.;
b) ≥15 wt. %, preferably ≥20 wt. %, of at least one odorant having a CLogP of ≥4.0 and a boiling point of ≤275° C.; and
c) ≥30 wt. %, preferably ≥40 wt. %, of at least one odorant having a vapor pressure of ≥5 Pa at 20° C.

The CLogP value is the liquid-liquid partition coefficient for the n-octanol-water system and a measure of the ratio between the lipophilicity and hydrophilicity of a substance. A value of greater than 1 denotes a rather lipophilic substance, a value below 1 denotes a substance which is more soluble in water than in n-octanol. The ClogP value can be calculated for any substance using suitable programs that are commercially available. Unless stated otherwise, the values specified herein are determined using the program EPI SUITE™ (v4.11) with the module KOWWIN™ v1.68.

Unless stated otherwise, the boiling point was determined using the program EPI SUITE™ (v4.11) with the module MPBPWIN v.1.43 (adapted Stein and Brown Method).

Unless stated otherwise, the vapor pressure at 20° C. was determined using the program EPI SUITE™ (v4.11) with the module MPBPWIN v.1.43 (modified grain method).

Examples of the fragrances of group a) include, but are not limited to:

| Odorant | CAS Number | CLogP | Boiling point [° C.] |
|---|---|---|---|
| Dulcinyl | 55418-52-5 | 2.03 | 295.74 |
| Furanol methyl ether | 4077-47-8 | 0.62 | 214.32 |
| Methyl corylone | 13494-06-9 | −0.12 | 219.34 |
| Tonkanyle ii | 874-90-8 | 1.62 | 230.92 |
| 2-Amino-1,3-propanediol serinol | 534-03-2 | −1.55 | 216.16 |
| 2.3-Diethyl pyrazine | 15707-24-1 | 2.02 | 209.41 |
| 3-(Methylthio)-1-hexanol | 51755-66-9 | 1.84 | 225.06 |
| Acetanisole | 100-06-1 | 1.75 | 229.45 |
| Acetate pa | 7493-74-5 | 2.46 | 268.49 |
| Aldehyde c 18 sog | 104-61-0 | 2.08 | 265.5 |
| Allyl amyl glycolate | 67634-00-8 | 2.34 | 217.66 |
| Anisaldehyde | 123-11-5 | 1.79 | 221.63 |
| Anisyl acetate | 104-21-2 | 2.16 | 252.81 |
| Benzyl acetate | 140-11-4 | 2.08 | 215.57 |
| Benzyl acetone | 2550-26-7 | 1.96 | 228.74 |
| Benzyl alcohol | 100-51-6 | 1.08 | 205.65 |
| Bicyclononalactone | 4430-31-3 | 1.55 | 271.33 |
| Birolan | 25322-68-3 | −2.3 | 353.51 |
| Buccoxime | 75147-23-8 | 2.2 | 264.34 |
| Calone | 28940-11-6 | 2.43 | 296.5 |
| Corps eglantine | 64988-06-3 | 2.35 | 231.03 |
| Corylone | 80-71-7 | 1.29 | 241.48 |
| Coumarin | 91-64-5 | 1.51 | 290.74 |
| Dmbc | 100-86-7 | 2.44 | 228.78 |
| Ethyl fruitate | 6413-10-01 | 1.3 | 217.74 |
| Ethyl maltol | 4940-11-08 | 0.3 | 283.37 |
| Ethyl vanillin | 121-32-4 | 1.55 | 290.04 |
| Florol | 63500-71-0 | 2.16 | 229.55 |
| Frambinone methyl ether | 104-20-1 | 2.04 | 264.7 |
| Guaiacol | 90-05-1 | 1.34 | 211.43 |
| Heliotropin | 120-57-0 | 1.77 | 256.96 |
| Homofuronol | 27538-09-6 | 1.31 | 275.21 |
| Hydratropic ald. dim. acetal | 90-87-9 | 2.35 | 227.23 |
| Hydratropic aldehyde | 93-53-8 | 1.96 | 209.32 |
| Hydroxycitronellal pure | 107-75-5 | 2.11 | 241.19 |
| Hydrocinnamic alcohol | 122-97-4 | 2.06 | 243.15 |
| Indocolore | 2206-94-2 | 2.49 | 227.9 |
| Indoflor | 18096-62-3 | 1.84 | 264.36 |
| Indole | 120-72-9 | 2.05 | 250.04 |
| Isobutavan | 20665-85-4 | 2.3 | 314.04 |
| Koumalactone | 92015-65-1 | 1.89 | 276.66 |
| Cresyl acetate (para) | 140-39-6 | 1.14 | 215.56 |
| Liffarome | 67633-96-9 | 2.47 | 213 |
| Linalool oxide | 1365-19-1 | 2.08 | 232.9 |
| Maltol | 118-71-8 | −0.19 | 267.24 |
| Malyl isobutyrate | 65416-14-0 | 1.7 | 286.06 |
| Menoxaline | 13708-12-8 | 1.66 | 265.62 |
| Methoxymelonal | 62439-41-2 | 2.32 | 205.16 |
| Methyl cinnamic alcohol | 1504-55-8 | 2.39 | 261.14 |
| Methyl diantilis | 5595-79-9 | 1.61 | 282.35 |
| Methyl octalactone | 39212-23-2 | 2 | 260.63 |
| Methyl acetophenone para | 122-00-9 | 2.22 | 209.72 |
| Methyl anthranilate | 134-20-3 | 2.26 | 263.57 |
| Methyl cinnamate | 103-26-4 | 2.36 | 239.9 |
| Methyl phenylacetate | 101-41-7 | 2.08 | 215.57 |
| Methyl serinol | 115-69-5 | −1.1 | 227.56 |
| Methyl cinnamic aldehyde (alpha) | 101-39-3 | 2.37 | 240.02 |
| Octalactone delta | 698-76-0 | 1.59 | 249.98 |
| Octalactone gamma | 104-50-7 | 1.59 | 248.37 |
| Orcinyl 3 | 3209-13-0 | 2.14 | 230.36 |
| Oxane 969380 | 59323-76-1 | 2.35 | 209.28 |
| Oxyphenylone | 5471-51-2 | 1.48 | 280.39 |
| Phenoxyethyl alcohol | 122-99-6 | 1.1 | 243.84 |
| Phenylacetaldehyde | 122-78-1 | 1.54 | 201.51 |
| Phenylacetaldehyde glycerine acetal | 29895-73-6 | 0.77 | 312.53 |
| Phenylacetaldehyde dimethyl acetal | 101-48-4 | 1.93 | 219.76 |
| Phenylacetic acid | 103-82-2 | 1.43 | 266.58 |
| Phenylethyl alcohol | 60-12-8 | 1.57 | 224.85 |
| Phenylethyl formate | 104-62-1 | 2.02 | 217.34 |
| Plicatone | 41724-19-0 | 2.45 | 259.49 |
| Propyl fruitate | 6290-17-1 | 1.72 | 231.07 |
| Styrolyl acetate | 93-92-5 | 2.5 | 223.12 |
| Sulfurol (4-methyl-5-thiazole ethanol) | 137-00-8 | 1.11 | 259.88 |
| Sulfurol milky | 137-00-8 | 1.11 | 259.88 |
| Toluylaldehyde (para) | 104-87-0 | 2.26 | 201.5 |
| Trifernal | 16251-77-7 | 2.45 | 228.35 |
| Vanillin | 121-33-5 | 1.05 | 274.3 |
| Veticol acetate | 68083-58-9 | 1.68 | 275.57 |
| Cinnamic aldehyde | 104-55-2 | 1.82 | 226.69 |
| Cinnamic alcohol | 104-54-1 | 1.84 | 248.6 |
| Diantheme | 4125-43-3 | 2.48 | 229.65 |
| Hydratropic alcohol | 1123-85-9 | 1.98 | 232.23 |
| Hydroxycitronellal synth. | 107-75-5 | 2.11 | 241.19 |
| Salicylaldehyde | 90-02-8 | 2.01 | 239.42 |
| Trivalon | 71566-51-3 | 1.39 | 241.44 |
| Anise alcohol | 105-13-5 | 1.16 | 243.83 |

Examples of fragrances of group b) include, but are not limited to:

| Odorant | CAS Number | CLogP | Boiling point [° C.] |
|---|---|---|---|
| aldehyde c 11 | 112-44-7 | 4.25 | 234.81 |
| aldehyde c 11.(en) | 112-45-8 | 4.12 | 233.44 |
| aldehyde c 12 | 112-54-9 | 4.75 | 252.62 |
| aldehyde c 12.mna | 110-41-8 | 4.67 | 241.99 |
| aldehyde supra | 143-14-6 | 4.04 | 240.39 |
| alcohol c 12 | 112-53-8 | 4.77 | 272.96 |
| allyl cyclohexyl propionate | 2705-87-5 | 4.47 | 254.19 |
| azarbre | 68845-36-3 | 4.11 | 259.5 |
| boisiris | 68845-00-1 | 5.44 | 256.67 |
| caryophyllene beta | 87-44-5 | 6.3 | 256.8 |
| cedramber | 19870-74-7 | 5.03 | 265.3 |
| cetonal | 73398-85-3 | 5.2 | 270.68 |
| cis-3-hexenyl-2-methylbutyrate | 53398-85-9 | 4.01 | 224.17 |
| citronellyl butyrate | 141-16-2 | 5.54 | 272.03 |
| citronellyl formate | 105-85-1 | 4.01 | 220.77 |
| citronellyl isobutyrate | 97-89-2 | 5.47 | 262.03 |
| citronellyl acetate | 150-84-5 | 4.56 | 237.59 |
| citronellyl propionate | 141-14-0 | 5.05 | 255.26 |
| coniferan pure | 67874-72-0 | 4.91 | 250.47 |
| cyclomyral | 68991-96-8 | 4.38 | 271.8 |
| cymol (para) = cymene | 99-87-6 | 4 | 178.34 |
| damarose alpha | 43052-87-5 | 4.29 | 259.84 |
| damascenone bm | 23696-85-7 | 4.21 | 265.13 |
| damascenone | 23696-85-7 | 4.21 | 265.13 |
| damascone alpha | 43052-87-5 | 4.29 | 259.84 |
| damascone beta | 35044-68-9 | 4.42 | 262.93 |
| decyl acetate | 112-17-4 | 4.79 | 247.73 |
| dihydro-beta-ionone | 17283-81-7 | 4.35 | 257.7 |
| dihydro floriffone td | 57378-68-4 | 4.16 | 255.99 |
| dihydro terpinyl acetate = menthanyl acetate | 58985-18-5 | 4.42 | 232.55 |
| diphenyl ether | 101-84-8 | 4.05 | 269.66 |
| dynascone pure | 56973-85-4 | 4.45 | 257.9 |
| ethyl damascenate | 35044-59-8 | 4.25 | 247.88 |
| ethyl-2-4-decadienoate | 3025-30-7 | 4.36 | 258.41 |
| ethyl linalyl acetate | 61931-80-4 | 4.88 | 247.05 |
| ethyl safranate | 35044-59-8 | 4.25 | 247.88 |
| fleuramone | 137-03-1 | 4 | 263.53 |
| floral super | 71077-31-1 | 4.38 | 241.28 |
| floramat | 67801-64-3 | 4.77 | 228.23 |
| folenox | 26619-69-2 | 4.38 | 247.81 |
| frutonile | 69300-15-8 | 4.2 | 250.43 |
| galbanolene super | 16356-11-9 | 5.18 | 196.6 |
| galbascone bht captive | 56973-85-4 | 4.45 | 257.9 |
| geraldehyde | 762-26-5 | 4.43 | 253.84 |
| geranyl isobutyrate | 2345-26-8 | 5.38 | 272.6 |
| geranyl propionate | 105-90-8 | 4.97 | 266.06 |
| givescone | 57934-97-1 | 4.83 | 259.38 |
| gyrane | 24237-00-1 | 4.07 | 214.86 |
| herbanate | 116044-44-1 | 4.13 | 254.37 |
| herbavert | 67583-77-1 | 4.12 | 193.28 |
| hexyl hexanoate | 6378-65-0 | 4.79 | 247.73 |
| irone alpha | 79-69-6 | 4.71 | 271.32 |
| isoamyl hexanoate | 2198-61-0 | 4.23 | 218.34 |
| isobornyl isobutyrate | 85586-67-0 | 4.77 | 251.13 |
| isodamascone | 33673-71-1 | 4.29 | 259.84 |
| isononyl acetate (neononyl acetate) | 58430-94-7 | 4.12 | 198.85 |
| ionone beta synth. | 14901-07-6 | 4.42 | 262.93 |
| ionone pure 100 | 127-41-3 | 4.29 | 259.48 |
| koavone | 86115-11-9 | 4.19 | 207.93 |
| linalyl acetate | 115-95-7 | 4.39 | 228.95 |
| mandarin aldehyde | 20407-84-5 | 4.53 | 257.92 |
| melusate | 67707-75-9 | 4.12 | 198.85 |
| menthyl acetate-rf | 89-48-5 | 4.39 | 234.5 |
| mercapto-8-menthene-1 para | 71159-90-5 | 4.74 | 224.11 |
| methyl ionone gamma coeur #1 | 127-51-5 | 4.84 | 271.6 |
| methyl octyl acetaldehyde (moa) | 19009-56-4 | 4.18 | 223.64 |
| myrcenyl acetate | 1118-39-4 | 4.47 | 221.78 |
| neobutenone alpha | 56973-85-4 | 4.45 | 257.9 |
| neryl acetate | 141-12-8 | 4.48 | 248.97 |
| neryl propionate | 105-91-9 | 4.97 | 266.06 |
| nopyl acetate | 128-51-8 | 4.3 | 259.16 |
| ocimenyl acetate | 72214-23-4 | 4.39 | 228.95 |
| otbca | 88-41-5 | 4.42 | 232.55 |
| ozofleur | 181258-89-9 | 5.1 | 228.54 |
| peranat | 90397-38-9 | 4.65 | 225.84 |
| phellandrene alpha extra | 99-83-2 | 4.62 | 165.01 |
| phenyl ethyl isoamyl ether | 56011-02-0 | 4.16 | 255.27 |
| pinane | 473-55-2 | 4.35 | 149.9 |
| pinene beta | 127-91-3 | 4.35 | 150.8 |
| pinene alpha | 80-56-8 | 4.27 | 157.25 |
| poirenate | 2511-00-4 | 4.04 | 226.74 |
| poivrol | 68966-86-9 | 4.62 | 259.95 |
| ptbca 25 cis | 32210-23-4 | 4.42 | 232.55 |
| ptbca 40 cis | 32210-23-4 | 4.42 | 232.55 |
| sandalore | 65113-99-7 | 5.15 | 273.81 |
| tangerinal 10 dipg semi-captive | 21944-98-9 | 4.53 | 257.92 |
| terpinene gamma | 99-85-4 | 4.75 | 169.36 |
| terpinene alpha p&f | 99-86-5 | 4.75 | 169.36 |
| terpinolene | 586-62-9 | 4.88 | 178.17 |
| terinyl methyl ether 90 | 14576-08-0 | 4.03 | 196.38 |
| tetrameran | 1322-58-3 | 4.44 | 250.01 |
| thesaron | 22471-55-2 | 4.42 | 235.87 |
| trithioacetone | 828-26-2 | 7.6 | 269.84 |
| troenan | 80480-24-6 | 4.35 | 253.14 |
| undecavertol | 81782-77-6 | 4.05 | 240.58 |
| undecylenic alcohol alcohol c 11 en | 112-43-6 | 4.14 | 254.95 |
| veloutone | 65443-14-3 | 4.34 | 257.68 |
| violette feuilles non dec. | 06.08.8024 | 4.29 | 259.48 |
| violettyne mip 991805 | 166432-52-6 | 4.68 | 204.87 |

Examples of fragrances of group c) include, but are not limited to:

| Odorant | CAS Number | Vapor pressure [Pa] 20° C. |
|---|---|---|
| linalool | 78-70-6 | 6,946 |
| peelessenz | 78-70-6 | 6,946 |
| dihydromyrcenol | 53219-21-9 | 6,986 |
| indocolore semi-captive | 2206-94-2 | 7,586 |
| ethyl phenyl acetate | 101-97-3 | 7,946 |
| citral ar | 5392-40-5 | 7,946 |
| citral n | 5392-40-5 | 7,946 |
| menthyl acetate-rf | 89-48-5 | 7,946 |
| poirenate | 2511-00-4 | 8,066 |
| ethyl acetate | 141-78-6 | 10265 |
| livescone | 3720-16-9 | 10,292 |
| decenal-4-trans | 65405-70-1 | 10,385 |
| myrcenyl acetate | 1118-39-4 | 10,492 |
| orivone | 16587-71-6 | 10,732 |
| methyl heptine carbonate | 111-12-6 | 11,052 |
| citronellyl formate | 105-85-1 | 11,065 |
| ozofleur | 181258-89-9 | 11,479 |
| carvone I | 6485-40-1 | 11,505 |
| linalyl acetate | 115-95-7 | 11,519 |
| benzyl propionate | 122-63-4 | 11,519 |
| montaverdi | 188570-78-7 | 11,585 |
| phenyl acetaldehyde dimethyl acetal | 101-48-4 | 11,665 |
| firascone | 815580-59-7 | 11,692 |
| delphone | 4819-67-4 | 11,705 |
| cyclohexyl ethyl acetate | 21722-83-8 | 11,985 |
| sclareolate | 319002-92-1 | 12,079 |
| centifolether | 120811-92-9 | 12,132 |
| bergamal | 22418-66-2 | 12,998 |
| allyl amyl glycolate | 67634-00-8 | 13,025 |
| phenyl ethyl formate | 104-62-1 | 13,252 |
| mintonat | 67859-96-5 | 13,332 |
| neofolione | 111-79-5 | 13,732 |
| cis-3-hexenyl butyrate | 16491-36-4 | 13,732 |
| methyl phenyl acetate | 101-41-7 | 13,865 |
| 9-decenal | 39770-05-3 | 15,198 |
| isocyclocitral | 1335-66-6 | 15,465 |
| benzyl acetate | 140-11-4 | 16,665 |
| aphermate | 25225-08-5 | 16,665 |
| liffarome | 67633-96-9 | 16,665 |
| cresyl acetate (para) | 140-39-6 | 17,065 |
| linalyl formate | 115-99-1 | 17,731 |
| dihydro anethol | 104-45-0 | 17,998 |
| freskomenthe | 14765-30-1 | 19,065 |
| octyl acetate | 112-14-1 | 19,465 |
| allyl heptanoate - allyl oenanthate | 142-19-8 | 20,265 |
| amyl vinyl carbinol | 3391-86-4 | 20,398 |
| nonadienal | 557-48-2 | 20,798 |
| ethyl caprylate | 106-32-1 | 21,064 |
| aldehyde c 10 | 112-31-2 | 21,064 |
| hexyl butyrate | 2639-63-6 | 21,598 |
| citronellal | 106-23-0 | 22,664 |
| gyrane | 24237-00-1 | 22,931 |
| claritone | 74338-72-0 | 24,797 |
| methoxymelonal | 62439-41-2 | 24,931 |
| hexenyl-cis-3-isobutyrate | 41519-23-7 | 25,331 |
| toluylaldehyde (para) | 104-87-0 | 25,864 |
| hydratropic aldehyde | 93-53-8 | 27,197 |
| acetophenone | 98-86-2 | 29,464 |
| 2-isopropyl-4-methylthiazole | 15679-13-7 | 29,864 |
| dimetol | 13254-34-7 | 31,597 |
| sultanene captive | 15848-49-4 | 31,597 |
| koavone | 86115-11-9 | 32,264 |
| methyl benzoate | 93-58-3 | 34,263 |
| damascia (frutinate) | 35206-51-0 | 46,796 |
| tetrahydrocitral | 5988-91-0 | 48,129 |
| phenyl ethyl methyl ether | 3558-60-9 | 50,129 |
| cuminaldehyde | 122-03-2 | 50,529 |
| aldehyde c 09 | 124-19-6 | 51,462 |
| dimethyl sulfide | 75-18-3 | 52129 |
| aldehyde c 11 | 112-44-7 | 52,395 |
| amyl valerianate, iso-iso- | 659-70-1 | 53,062 |
| tetrahydrolinalool | 78-69-3 | 53,728 |
| lilessenz | 78-69-3 | 53,728 |
| octalactone gamma | 104-50-7 | 54,662 |
| galbanolene super | 16356-11-9 | 56,128 |
| benzyl acetone | 2550-26-7 | 56,128 |
| aldehyde c 11.(en) | 112-45-8 | 56,395 |
| toscanol | 16510-27-3 | 56,395 |
| otbca | 88-41-5 | 56,662 |
| agrunitrile | 51566-62-2 | 57,195 |
| methyl pamplemousse | 67674-46-8 | 57,861 |
| amarocite | 67674-46-8 | 57,861 |
| geranyl formate | 105-86-2 | 58,795 |
| phenyl ethyl acetate | 103-45-7 | 58,928 |
| methyl octine carbonate | 111-80-8 | 59,061 |
| ptbca 40 cis | 32210-23-4 | 59,195 |
| ptbca 25 cis | 32210-23-4 | 59,195 |
| dihydro terpinyl acetate = menthanyl acetate | 58985-18-5 | 59,195 |
| fenchyl acetate | 13851-11-1 | 59,328 |
| velberry | 13431-57-7 | 59,999 |
| rose oxide r | 16409-43-1 | 60,795 |
| rose oxide l | 3033-23-6 | 60,795 |
| propyl fruitate | 6290-17-1 | 61,061 |
| ethyl heptanoate | 106-30-9 | 63,061 |
| corps eglantine | 64988-06-3 | 64,128 |
| isoamyl-2-methylbutyrate | 27625-35-0 | 64,261 |
| 2.3 diethylpyrazine | 15707-24-1 | 64,528 |
| irolene p | 29127-83-1 | 64,661 |
| dimethyl octenone | 2550-11-0 | 65,327 |
| cyclopidene | 40203-73-4 | 65,327 |
| allyl capronate | 123-68-2 | 66,261 |
| methyl acetophenone para | 122-00-9 | 67,194 |
| hydroquinone dimethyl ether | 150-78-7 | 67,194 |
| 2-cyclopentyl cyclopentanone | 4884-24-6 | 68,394 |
| dibutyl sulphide | 544-40-1 | 69,727 |
| ocimenyl acetate | 72214-23-4 | 71,727 |
| trifernal | 16251-77-7 | 73,993 |
| 4-methylthio-4-methyl-2-pentanone | 23550-40-5 | 81,326 |
| citrathal | 147060-73-9 | 82,659 |
| citral dimethyl acetal | 7549-37-3 | 82,659 |
| alcohol c 08 | 111-87-5 | 83,059 |
| hexenol (beta gamma) | 928-96-1 | 83,459 |
| octanol-3 | 589-98-0 | 83,593 |
| isobornyl acetate | 125-12-2 | 85,059 |
| ethyl acetoacetate | 141-97-9 | 85,992 |
| givescone | 57934-97-1 | 87,326 |
| guaiacol | 90-05-1 | 88,526 |
| isopentyrate | 80118-06-5 | 91,992 |
| terpinolene | 586-62-9 | 93,592 |
| benzaldehyde pure | 100-52-7 | 93,992 |
| methyl octyl acetaldehyde | 19009-56-4 | 95,058 |
| vivaldie | 292605-05-1 | 95,458 |
| tetrahydromyrcenol | 18479-57-7 | 95,725 |
| styrolyl acetate | 93-92-5 | 97,725 |
| methyl nonyl ketone | 112-12-9 | 98,791 |
| hexenyl acetate | 3681-71-8 | 106.12 |
| terpinene gamma | 99-85-4 | 108.12 |
| cresyl methyl ether (para) | 104-93-8 | 111.85 |
| orrisate | 816-19-3 | 133.32 |
| hexyl acetate | 142-92-7 | 135.98 |
| aldehyde c 12.mna | 110-41-8 | 139.98 |
| aldehyde c 08 | 124-13-0 | 139.98 |
| myroxide | 69103-20-4 | 143.98 |
| ethyl butyrate | 105-54-4 | 1453.2 |
| 2.6-dimethyl pyrazine | 108-50-9 | 147.98 |
| amyl propionate | 624-54-4 | 157.32 |
| butyl butyrate | 109-21-7 | 165.31 |
| methyl heptenone | 110-93-0 | 169.31 |
| ethyl capronate | 123-66-0 | 169.31 |
| methyl hexyl ketone | 111-13-7 | 177.31 |
| acetoin | 513-86-0 | 181.31 |
| phellandrene alpha extra | 99-83-2 | 181.31 |
| isobutyl acetate | 110-19-0 | 1826.5 |
| limetol | 7392-19-0 | 191.98 |
| cerezoate | 1617-23-8 | 198.65 |
| neoproxen | 31996-78-8 | 198.65 |
| cyclamen aldehyde extra I.g. | 103-95-7 | 2053.1 |
| pinane | 473-55-2 | 209.31 |
| isoamyl propionate | 105-68-0 | 235.98 |
| pinene beta p&f | 127-91-3 | 239.98 |
| ethyl isobutyrate | 97-62-1 | 2439.7 |
| hexenyl-3-formate | 33467-73-1 | 273.31 |
| methylbutyl-2 propionate | 2438-20-2 | 274.64 |
| manzanate | 39255-32-8 | 274.64 |
| phenyl acetaldehyde | 122-78-1 | 28.93 |
| ethyl tiglate | 5837-78-5 | 289.3 |
| triplal | 27939-60-2 | 31.73 |
| diethyl malonate | 105-53-3 | 32.53 |
| menthone | 14073-97-3 | 33.73 |
| isomenthone | 491-07-6 | 33.73 |
| ethyl amyl ketone | 541-85-5 | 331.97 |
| aldehyde c 07 | 111-71-7 | 337.3 |
| isononyl acetate (neononyl acetate) | 58430-94-7 | 34.53 |
| rumacetal | 1670-47-9 | 36.13 |
| 2-isobutyl thiazole | 18640-74-9 | 36.13 |
| pinene alpha | 80-56-8 | 390.63 |
| prenyl acetate | 1191-16-8 | 401.3 |
| isobutyl isobutyrate | 97-85-8 | 411.96 |
| hexenal-2-trans | 6728-26-3 | 455.96 |
| ethyl valerate | 539-82-2 | 463.96 |
| isopropyl methyl-2-butyrate | 66576-71-4 | 517.29 |
| isovaleraldehyde | 590-86-3 | 5306.2 |
| melonal | 106-72-9 | 69.86 |
| hexanol | 111-27-3 | 78.26 |
| ethyl-2-methylbutyrate | 7452-79-1 | 786.6 |
| aldehyde c 06 | 66-25-1 | 941.25 |

Preferably, mixtures of odorants are used, for example at least two or more different odorants of groups b) and/or at least two or more different odorants of group c).

In various embodiments of the invention, the proportion of the odorants of group a) may also be less than 6, less than 5, less than 4, less than 3, less than 2, or less than 1 wt. %. In various embodiments, the perfume composition may contain no odorants of group a). In alternative embodiments, the perfume composition contains odorants of group a), but in amounts below the upper limits indicated here.

In various embodiments, the fragrances of groups b) and c) may together make up at least 60, preferably at least 65, at least 70, at least 75, at least 80, at least 85, or at least 90 wt. % of the total odorants of the perfume composition.

The quantitative data given herein refer to the sum of all odorants in the encapsulated perfume composition, unless otherwise indicated. Alternatively, they may also refer to the total weight of the encapsulated perfume composition, for example when same contains formulation auxiliaries.

In addition to the above-mentioned fragrances of groups a) to c), further fragrances may be used as constituents of the encapsulated perfume composition, as long as features a) to c) are met. These additional fragrances are not subject to any particular restrictions. Thus, individual fragrance compounds of natural or synthetic origin, for example of the ester, ether, aldehyde, ketone, alcohol and hydrocarbon type, can be used. The core materials may also contain natural fragrance mixtures, such as those that can be obtained from plant sources, for example pine, citrus, jasmine, patchouli, rose, or ylang-ylang oil. Equally, clary sage oil, chamomile oil, clove oil, melissa oil, mint oil, cinnamon leaf oil, lime blossom oil, juniper berry oil, vetiver oil, olibanum oil, galbanum oil, and labdanum oil, as well as orange blossom oil, neroli oil, orange peel oil, and sandalwood oil are also suitable.

The tightness of the capsule wall can be influenced by the choice of shell components. According to one embodiment, the microcapsules have a tightness which ensures egress of at most 45 wt. %, at most 40 wt. %, at most 35 wt. %, at most 30 wt. %, at most 25 wt. %, at most 20 wt. % of the core material used during storage over a period of 4 weeks at a temperature of 0 to 40° C. In various embodiments, the microcapsules still contain at least 55 wt. %, preferably at least 60 wt. %, more preferably at least 65 wt. %, even more preferably at least 70 wt. %, even more preferably at least 75 wt. %, even more preferably at least 80 wt. %, of the core material used during storage over a period of 4 weeks at a temperature of 0 to 40° C.

The microcapsules are stored in a model formulation that corresponds to the target application. Moreover, the microcapsules are also storage-stable in the product in which they are used. For example, in detergents, fabric softeners, or cosmetic products. The guideline formulations for these products are known to a person skilled in the art. Typically, the pH in the environment of the microcapsules is in the range of from 2 to 12 during storage.

The microcapsule shells according to the invention have at least two layers, i.e., they can have, for example, two layers, three layers, four layers or five layers. The microcapsules preferably have two or three layers.

According to one embodiment, the microcapsule has a third layer which is arranged on the outer side of the stability layer. This third layer can be used to adapt the surface properties of the microcapsule for a particular application. These include an improvement of the adhesion of the microcapsules to a wide variety of surfaces and a reduction of the agglomeration. The third layer also binds residual quantities of aldehyde, thus reducing the content of free aldehydes in the capsule dispersion. Furthermore, it can provide additional (mechanical) stability or further increase the tightness. Depending on the application, the third layer may contain a component selected from amines, organic salts, inorganic salts, alcohols, ethers, polyphosphazenes, and noble metals.

Noble metals increase the tightness of the capsules and can give the microcapsule surface additional catalytic properties or the antibacterial action of a silver layer. Organic salts, in particular ammonium salts, lead to cationization of the microcapsule surface, which results in the microcapsule surface adhering better to textiles, for example. Alcohols also lead to the formation of H bridges when integrated via free hydroxyl groups, which also allow for better adhesion to substrates. An additional polyphosphazene layer or a coating of inorganic salts, for example silicates, leads to an additional increase in the tightness without affecting the biodegradability. According to a preferred embodiment, the third layer contains activated melamine. The melamine absorbs possible free aldehyde fractions of the stability and/or barrier layer, increases the tightness and stability of the capsule, and can additionally influence the surface properties of the microcapsules and thus the adhesion and agglomeration behavior.

Due to the small wall thicknesses, the proportion of the barrier layer in the shell relative to the total weight of the shell is at most 30 wt. %. The proportion of the barrier layer in the shell relative to the total weight of the shell may, for example, be 30 wt. %, 28 wt. %, 25 wt. %, 23 wt. %, 20 wt. %, 18 wt. %, 15 wt%, 13 wt. %, 10 wt. %, 8 wt. %, or 5 wt. %. . . For high biodegradability, the proportion is at most 25 wt. % based on the total weight of the shell. Particularly preferably, the proportion of the barrier layer is at most 20 wt. %. The proportion of the stability layer in the shell relative to the total weight of the shell is at least 40 wt. %. The proportion of the stability layer in the shell relative to the total weight of the shell may, for example, be 40 wt. %, 43 wt. %, 45 wt. %, 48 wt. %, 50 wt. %, 53 wt. %, 55 wt. %, 58 wt. %, 60 wt. %, 63 wt. %, 65 wt. %, 68 wt. %, 70 wt. %, 75 wt. %, 80 wt. %, 85 wt. %, or 90 wt. %. .. For a high biodegradability, the proportion of the stability layer is at least 50 wt. %, particularly preferably at least 60 wt. %. The proportion of the third layer in the shell relative to the total weight of the shell is at most 35 wt. %. The proportion of the third layer in the shell relative to the total weight of the shell may, for example, be 35 wt. %, 33 wt. %, 30 wt. %, 28 wt. %, 25 wt. %, 23 wt. %, 20 wt. %, 18 wt. %, 15 wt. %, 13 wt. %, 10 wt. %, 8 wt. %, or 5 wt. %. . . For a high biodegradability, the proportion of the third layer is at most 30 wt. %, particularly preferably at most 25 wt. %.

The size of the microcapsules according to the invention is in the range customary for microcapsules. The diameter can be in the range from 100 nm to 1 mm. The diameter is dependent on the exact capsule composition and the preparation method. The peak maximum of the particle size distribution is regularly used as the characteristic value for the size of the capsules. The peak maximum of the particle size distribution is preferably in the range of from 1 μm to 500 μm. The peak maximum of the particle size distribution may, for example, be 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, 120 μm, 140 μm, 160 μm, 180 μm, 200 μm, 250 μm, 300 μm, 350 μm, 400 μm, 450 μm, or 500 μm. According to a particularly preferred embodiment, the microcapsules have a peak maximum of the particle size distribution of 10 μm to 100 μm. In particular, the peak maximum of the particle size distribution is in the range from 10 μm to 50 μm.

The use of the emulsion stabilizer for coating the barrier layer constitutes a novel application that should be distinguished from the conventional use of the emulsion stabilizer, namely the stabilization of the core material droplets.

### Detergent or Cleaning Agent Containing Microcapsules

Due to the robustness or tightness of these biodegradable capsules, they can advantageously be used in a detergent and cleaning agent or in cosmetic agents, these agents including fabric softeners, textile care agents, solid detergents, for example granules or powders, liquid detergents, household cleaners, bath and toilet cleaners, hand dishwashing agents, machine dishwashing agents, hand soaps, shampoos, shower gels, creams, and the like, but are not limited thereto.

The washing or cleaning agents of the invention preferably comprise at least one ingredient selected from the group consisting of surfactants, enzymes, builders, and agents that enhance absorption.

The washing and cleaning agents can also contain anionic, nonionic, cationic, amphoteric or zwitterionic surfactants or mixtures thereof. Furthermore, these agents can be present in solid or liquid form. In various embodiments, the surfactants comprise at least one anionic surfactant and/or at least one nonionic surfactant.

Suitable non-ionic surfactants are, in particular, ethoxylation and/or propoxylation products of alkyl glycosides and/or linear or branched alcohols each having 12 to 18 C atoms in the alkyl moiety and 3 to 20, preferably 4 to 10 alkyl ether groups. Furthermore, corresponding ethoxylation and/or propoxylation products of N-alkylamines, vicinal diols, fatty acid esters, and fatty acid amides, which correspond to the above-mentioned long-chain alcohol derivatives with respect to the alkyl moiety, and of alkylphenols having 5 to 12 C atoms in the alkyl radical, may be used.

Suitable anionic surfactants are, in particular, soaps and those which contain sulfate or sulfonate groups, preferably having alkali ions as cations. Usable soaps are, preferably, alkali salts of saturated or unsaturated fatty acids having 12 to 18 C atoms. Fatty acids of this kind can also be used in a not completely neutralized form. Usable surfactants of the sulfate type include salts of sulfuric acid half-esters of fatty alcohols having 12 to 18 C atoms and sulfation products of the above-mentioned non-ionic surfactants having a low degree of ethoxylation. Usable surfactants of the sulfonate type include linear alkylbenzene sulfonates having 9 to 14 C atoms in the alkyl moiety, alkane sulfonates having 12 to 18 C atoms, and olefin sulfonates having 12 to 18 C atoms, which are formed in the reaction of corresponding monoolefins with sulfur trioxide, as well as alpha-sulfofatty acid esters, which are formed during the sulfonation of fatty acid methyl or ethyl esters.

Cationic surfactants are preferably selected from among esterquats and/or quaternary ammonium compounds (QAC) according to the general formula (R^{I})(R^{II})(R^{III})(R^{IV})N⁺ X⁻, in which R^{I} to R^{IV} stand for equal or different C₁₋₂₂ alkyl radicals, C₇₋₂₈ aryl alkyl radicals, or heterocyclic radicals, with two or, in the case of aromatic integration as in pyridine, even three radicals, together with the nitrogen atom, forming the heterocycle, e.g. a pyridinium or imidazolinium compound, and X⁻ standing for halide ions, sulfate ions, hydroxide ions, or similar anions. QACs can be prepared by reacting tertiary amines with alkylating agents, such as methyl chloride, benzyl chloride, dimethyl sulfate, dodecyl bromide, but also ethylene oxide. The alkylation of tertiary amines with one long alkyl radical and two methyl groups is particularly easy, and quaternization of tertiary amines with two long radicals and one methyl group can be carried out with the aid of methyl chloride under mild conditions. Amines having three long alkyl radicals or hydroxy-substituted alkyl radicals are less reactive and are quaternized with dimethyl sulfate, for example. Suitable QACs are, for example, benzalkonium chloride (N-alkyl-N,N-dimethyl benzyl ammonium chloride), benzalkone B (m,p-dichlorobenzyl dimethyl-C₁₂-alkylammonium chloride, benzoxonium chloride (benzyl dodecyl-bis-(2-hydroxyethyl)-ammonium chloride), cetrimonium bromide (N-hexadecyl-N, N-trimethyl ammonium bromide), benzethonium chloride (N,N dimethyl-N [2-[2-[p-(1, 1,3,3-tetramethylbutyl) phenoxy] ethoxy] ethyl] benzyl ammonium chloride), dialkyl dimethyl ammonium chlorides such as Di-n-decyl dimethyl ammonium chloride, didecyl dimethyl ammonium bromide, dioctyl dimethyl ammonium chloride, 1-cetyl pyridinium chloride, and thiazoline iodide, and mixtures thereof. Preferred QACs are benzalkonium chlorides having C₈₋₂₂ alkyl radicals, in particular C₁₂₋₁₄ alkyl benzyl dimethyl ammonium chloride.

Preferred esterquats are methyl-N-(2-hydroxyethyl)-N, N-di(talgacyloxyethyl)ammonium methosulfate, bis(palmitoyl)ethyl hydroxyethyl methyl ammonium methosulfate, or methyl-N,N-bis(acyloxyethyl)-N-(2-hydroxyethyl)ammonium methosulfate. Commercially available examples are the methylhydroxyalkyldialkoyloxyalkyl ammonium methosulfates marketed by Stepan under the trademark Stepantex®, the products from BASF SE known under the trade name Dehyquart®, or the products from Evonik known under the name Rewoquat®.

The amounts of the individual ingredients in the detergents and cleaning agents are each based on the intended purpose of the composition in question and a person skilled in the art is generally familiar with the orders of magnitude of the amounts of the ingredients to be used or can derive this from the relevant technical literature. Depending on the intended use of the compositions, the surfactant content, for example, is selected to be higher or lower. Typically, for example, the surfactant content of detergents may be from 10 to 50 wt. %, preferably from 12.5 to 30 wt. %, and more preferably from 15 to 25 wt. %.

The detergents and cleaning agents may contain, for example, at least one water-soluble and/or water-insoluble, organic and/or inorganic builder. The water-soluble organic builder substances include polycarboxylic acids, in particular citric acid and saccharic acids, monomer and polymer aminopolycarboxylic acids, in particular methylglycinediacetic acid, nitrilotriacetic acid, ethylenediaminetetraacetic acid, and polyaspartic acid, polyphosphonic acids, in particular aminotris(methylene phosphonic acid), ethylenediaminetetrakis(methylenephosphonic acid), and 1-hydroxyethane-1,1-diphosphonic acid, polymer hydroxy compounds such as dextrin, and also polymer (poly)carboxylic acids, polymer acrylic acids, methacrylic acids, maleic acids, and mixed polymers from these, which may also contain small proportions of polymerizable substances without carboxylic acid functionality in polymerized form. Suitable, albeit less preferred compounds of this class are copolymers of acrylic acid or methacrylic acid with vinyl ethers, such as vinyl methyl ethers, vinyl esters, ethylene, propylene, and styrene, in which the proportion of the acid is at least 50 wt. %. The organic builder substances can be used, in particular, for preparing liquid detergents and cleaning agents, in the form of aqueous solutions, preferably in the form of 30 to 50 wt. % aqueous solutions. All of said acids are generally used in the form of their water-soluble salts, in particular their alkali salts.

Organic builder substances may, if desired, be contained in amounts of up to 40 wt. %, in particular up to 25 wt. %, and preferably from 1 wt. % to 8 wt. %. Amounts close to the stated upper limit are preferably used in pasty or liquid, in particular water-containing, agents according to the invention. Laundry post-treatment agents, such as fabric softeners, may optionally also be free of organic builders.

In particular, alkali silicates and polyphosphates, preferably sodium triphosphate, are suitable as water-soluble inorganic builder materials. In particular, crystalline or amorphous alkali aluminosilicates may be used as water-insoluble, water-dispersible inorganic builder materials, if desired, in amounts of up to 50 wt. %, preferably not more than 40 wt. %, and in liquid compositions from 1 wt. % to 5 wt. %, in particular. Among these, crystalline sodium aluminosilicates of detergent quality, in particular zeolite A, P, and optionally X, are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm.

Suitable substitutes or partial substitutes for the above-mentioned aluminosilicate are crystalline alkali silicates, which can be present alone or in a mixture with amorphous silicates. The alkali silicates that can be used as builders in detergents or cleaning agents preferably have a molar ratio of alkali oxide to SiO₂ of under 0.95, in particular 1:1.1 to 1:12, and can be present in amorphous or crystalline form. Preferred alkali silicates are sodium silicates, in particular amorphous sodium silicates, having a molar ratio of Na₂O:SiO₂ of from 1:2 to 1:2.8. Crystalline sheet silicates of the general formula Na₂SiₓO₂ₓ₊₁·yH₂O, in which x, the so-called modulus, is a number from 1.9 to 4 and y is a number from 0 to 20 and preferred values for x are 2, 3, or 4, are preferably used as crystalline silicates, which can be present alone or in a mixture with amorphous silicates. Preferred crystalline sheet silicates are those in which x assumes the values 2 or 3 in the above-mentioned general formula. In particular, both beta- and delta-sodium disilicates (Na₂Si₂O₅·yH₂O) are preferred. Virtually anhydrous crystalline alkali silicates that are produced from amorphous alkali silicates and that are of the above-mentioned general formula, in which x is a number from 1.9 to 2.1, may also be used. In another preferred embodiment, a crystalline sodium sheet silicate having a modulus of 2 to 3 of the like that can be produced from sand and soda is used. Crystalline sodium silicates having a modulus in the range of from 1.9 to 3.5 are used in another preferred embodiment of the textile treatment or cleaning agents. If alkali aluminosilicate, in particular zeolite, is also present as an additional builder substance, the weight ratio of aluminosilicate to silicate, based in each case on anhydrous active substances, is preferably 1:10 to 10:1. In compositions containing both amorphous and crystalline alkali silicates, the weight ratio of amorphous alkali silicate to crystalline alkali silicate is preferably 1:2 to 2:1 and in particular 1:1 to 2:1.

Builders are preferably contained in amounts of up to 60 wt. %, in particular 5 wt. % to 40 wt. %, if desired. Laundry post-treatment agents, such as fabric softeners, are preferably free of inorganic builder.

In various embodiments, an agent according to the invention further comprises at least one enzyme.

The enzyme may be a hydrolytic enzyme or other enzyme in a concentration that is appropriate for the effectiveness of the agent. One embodiment of the invention thus constitutes agents comprising one or more enzymes. All enzymes which can exhibit catalytic activity in the agent according to the invention can preferably be used as enzymes, in particular a protease, amylase, cellulase, hemicellulase, mannanase, tannase, xylanase, xanthanase, xyloglucanase, β-glucosidase, pectinase, carrageenase, perhydrolase, oxidase, oxidoreductase, or a lipase, and mixtures thereof. Enzymes are advantageously each contained in the agent in an amount of from 1×10⁻⁸ to 5 wt. %, relative to active protein. In order of increasing preference, each enzyme is contained in agents according to the invention in an amount of from 1×10⁻⁷ to 3 wt. %, 0.00001 to 1 wt. %, 0.00005 to 0.5 wt. %, 0.0001 to 0.1 wt. %, and particularly preferably 0.0001 to 0.05 wt. %, relative to active protein. Particularly preferably, the enzymes exhibit synergistic cleaning performance with respect to particular dirt or stains, i.e. the enzymes contained in the agent composition assist one another in their cleaning performance. Synergistic effects can occur not only between different enzymes but also between one or more enzymes and other ingredients of the agent according to the invention.

The amylase(s) is/are preferably an a-amylase. The hemicellulase is preferably a pectinase, a pullulanase, and/or a mannanase. The cellulase is preferably a cellulase mixture or a single-component cellulase, preferably or predominantly an endoglucanase and/or a cellobiohydrolase. The oxidoreductase is preferably an oxidase, in particular a choline oxidase, or a perhydrolase.

The proteases used are preferably alkaline serine proteases. They act as unspecific endopeptidases, i.e. they hydrolyze any acid amide bonds that lie inside peptides or proteins and thereby cause the degradation of protein-containing dirt on the washware. Their optimum pH is usually in the distinctly alkaline range. In preferred embodiments, the enzyme contained in the agent according to the invention is a protease.

The enzymes used herein may be naturally occurring enzymes or enzymes that have been altered by one or more mutations based on naturally occurring enzymes in order to positively influence desired properties such as catalytic activity, stability, or disinfecting performance.

In preferred embodiments of the invention, the enzyme is contained in the agent according to the invention in the form of an enzyme product in an amount of from 0.01 to 10 wt. %, preferably 0.01 to 5 wt. %, relative to the total weight of the agent. The active protein content is preferably in the range of from 0.00001 to 1 wt. %, in particular 0.0001 to 0.2 wt. %, relative to the total weight of the agent.

The protein concentration can be determined using known methods, for example the BCA method (bicinchoninic acid; 2,2′-bichinolyl-4,4′-dicarboxylic acid) or the Biuret method. In this regard, the active protein concentration is determined by means of titration of the active centers using a suitable irreversible inhibitor (for proteases, for example, phenylmethylsulfonyl fluoride (PMSF)) and determination of the residual activity (cf. M. Bender et al., J. Chem. Soc. 88, 24 (1966), p.5890-5913).

In the agents described herein, the enzymes to be used may also be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These ready-made preparations include, for example, the solid preparations obtained by means of granulation, extrusion or lyophilization or, in particular in the case of liquid or gel agents, solutions of the enzymes, which are advantageously as concentrated as possible, have a low water content, and/or are admixed with stabilizers or further auxiliaries.

Alternatively, the enzymes can be encapsulated both for the solid and for the liquid administration form, for example by means of spray-drying or extrusion of the enzyme solution together with a preferably natural polymer or in the form of capsules, for example those in which the enzymes are enclosed as in a solidified gel, or in those of the core-shell type, in which an enzyme-containing core is coated with a protective layer that is impermeable to water, air and/or chemicals. Further active ingredients, for example, stabilizers, emulsifiers, pigments, bleaches or dyes can additionally be applied in overlaid layers. Such capsules are made using methods that are known per se, for example by means of vibratory granulation or roll granulation or by means of fluid bed processes. Advantageously, such granules are low in dust, for example due to the application of polymeric film formers, and are stable in storage due to the coating.

Furthermore, it is possible to formulate two or more enzymes together such that a single granule exhibits a plurality of enzyme activities.

In various embodiments, the agent according to the invention may have one or more enzyme stabilizers.

Attachment-enhancing agents are means which improve the attachment of the microcapsules to surfaces, in particular textile surfaces. This category of agents includes, for example, the esterquats mentioned above. Further examples are so-called SRPs (soil repellent polymers), which may be non-ionic or cationic, of note being, in particular, polyethylenimines (PEI) and ethoxylated variants thereof and polyesters, in particular esters of terephthalic acid, especially those of ethylene glycol and terephthalic acid or polyester/polyethers of polyethylene terephthalate and polyethylene glycol. Finally, anionic or non-ionic silicones also fall under this group. Exemplary compounds are also disclosed in patent specification EP 2 638 139 A1.

Furthermore, the detergents and cleaning agents may contain further ingredients which further improve the practical and/or aesthetic properties of the composition depending on the intended use. In the context of the present invention, they may contain bleaching agents, bleach activators, bleach catalysts, esterquats, silicone oils, emulsifiers, thickeners, electrolytes, pH adjusters, fluorescent agents, dyes, hydrotopes, foam inhibitors, anti-redeposition agents, solvents, optical brighteners, graying inhibitors, anti-shrinking agents, anti-crease agents, dye transfer inhibitors, color protection agents, wetting enhancers, antimicrobial active ingredients, germicides, fungicides, antioxidants, corrosion inhibitors, rinse aids, preservatives, antistatic agents, ironing aids, repellents and impregnation agents, pearlescent agents, swelling and antislip agents, and UV absorbers, without being limited thereto.

Suitable ingredients and framework compositions for detergent and cleaning agent compositions (for example for detergents and fabric softeners) are disclosed, for example, in EP 3 110 393 B1.

### Preparation Process

Methods for preparing core/shell microcapsules are known to a person skilled in the art. As a rule, an oil-based core material that is insoluble or poorly soluble in water is emulsified or dispersed in an aqueous phase containing the wall-forming agents. Depending on the viscosity of liquid core materials, a wide variety of units are used, from a simple stirrer to a high-performance disperser, which units distributes the core material into fine oil droplets. In the process, the wall formers are separated from the continuous water phase on the surface of the oil droplets and can subsequently be crosslinked.

This mechanism is used during in-situ polymerization of amino and phenoplast microcapsules and during coacervation of water-soluble hydrocolloids.

In contrast, during radical polymerization, oil-soluble acrylate monomers are used for the formation of wall. In addition, methods are used in which water-soluble and oil-soluble starting materials are reacted at the phase boundary of the emulsion droplets, which form the solid shell.

Examples thereof are the reaction of isocyanates and amines or alcohols to form polyurea or polyurethane walls (interfacial polymerization), but also the hydrolysis of silicate precursors with subsequent condensation, forming an inorganic capsule wall (sol-gel method).

In suitable methods for preparing microcapsules comprising a fragrance as the core material and a shell consisting of three layers, the barrier layer serving as a diffusion barrier is initially provided as a template. To construct this barrier layer, very small proportions of wall formers of the type mentioned are required. After the droplet formation at high stirring speeds, the sensitive templates are preferably provided with an electrically negative charge by means of suitable protective colloids (e.g. polyAMPS), such that neither Ostwald ripening nor coalescence can occur. After this stable emulsion has been prepared, for example a suitable precondensate based on aminoplast resin can form in a very thin shell (layer) with a greatly reduced stirring speed of the wall formers. The thickness of the shell can be further reduced, in particular by adding an aromatic alcohol, for example m-aminophenol. This is followed by the formation of a production-ready shell structure which unexpectedly shows a good affinity to biopolymers such as gelatin or alginate when same are added and is deposited on the templates without the expected problems such as gelling of the batch, agglomerate formation, and incompatibility of the structuring agent.

By using the emulsion stabilizer, the deposition of the biopolymers can be further increased, as shown in the examples.

The procedure comprises at least the following steps:
a) preparing an oil-in-water emulsion by emulsifying a core material in an aqueous phase in the presence of the wall-forming component(s) of the inner barrier layer with the addition of protective colloids;
b) depositing and curing the wall-forming component(s) of the barrier layer, the wall-forming component(s) of the barrier layer preferably being an aldehyde component, an amine component, and an aromatic alcohol, particularly preferably formaldehyde, melamine, and resorcinol;
c) adding an emulsion stabilizer, the emulsion stabilizer being as defined herein;
d) adding the wall-forming component(s) of the stability layer, followed by deposition and curing, the wall-forming component(s) of the stability layer being at least one biopolymer, preferably a protein and/or a polysaccharide, particularly preferably gelatin and alginate, and a curing agent, preferably glutaraldehyde or glyoxal; and
e) optionally adding the wall-forming component(s) of the outer, third shell layer, followed by deposition and curing, the wall-forming component(s) of the outer, third shell layer preferably being an amine component, in particular melamine.

In various process steps, the addition of a thickener, such as Jaguar HP105 (Solvay), can be advantageous. The thickening agent serves in particular to adjust the viscosity. An increase in viscosity, for example up to a viscosity of 2500 mPas (measured using Brookfield, RT, S3), can stabilize the microcapsule dispersion and thus improve the stirrability and storage.

The emulsion stabilizer is preferably added slowly over at least two minutes. According to one embodiment, the microcapsule dispersion is stirred. A paddle stirrer, for example, may be used for the stirring. The stirring speed is preferably in the range of from 150 to 250 rpm. Above 250 rpm, there is a risk of air being introduced into the microcapsule dispersion. Below 150 rpm, the mixing may not be sufficient.

The temperature is preferably in the range of from 15° C. to 35° C. The temperature may be 15° C., 18° C., 20° C., 23° C., 25° C., 28° C., 30° C., 33° C., or 35° C. The temperature is particularly preferably 25° C. After being added, the microcapsule dispersion is stirred until a homogeneous mixture is formed. In one embodiment, the microcapsule dispersion is stirred for at least 5 min after being added. In a preferred embodiment, the microcapsule dispersion is stirred for at least 10 min after being added.

Alternatively, steps a) and b) can be carried out as follows:
a) preparing an oil-in-water emulsion by emulsifying a core material in an aqueous phase in the presence of the wall-forming component(s) of the inner barrier layer, optionally with addition of protective colloids;
b) depositing and curing the wall-forming component(s) of the inner barrier layer, the wall-forming component(s) of the inner barrier layer preferably being an aldehyde component, an amine component, and an aromatic alcohol.

This method can be carried out either sequentially or as a so-called one-pot method. In the sequential method, in a first method, only steps a) and b) are carried out until microcapsules having only the inner barrier layer as the shell (intermediate microcapsules) are obtained. Subsequently, some or all of these intermediate microcapsules are then transferred into another reactor. The further reaction steps are then carried out therein. In the single-pot method, all method steps are carried out in a batch reactor. Performing the method without changing reactors is particularly time-saving.

For this purpose, the overall system should be tailored to the one-pot method. This allows for the correct choice of solid fractions, the correct temperature control, the tailored addition of formulation constituents, and the sequential addition of the wall formers.

In one embodiment of the method, the method comprises preparing a water phase by dissolving a protective colloid, in particular a polymer based on acrylamide sulfonate and a methylated prepolymer, in water. The prepolymer is preferably produced by reacting an aldehyde with either melamine or urea. Methanol can optionally be used here.

Furthermore, in the method according to the invention, the water phase can be thoroughly mixed by stirring and setting a first temperature, the first temperature being in the range from 30 to 40° C. An aromatic alcohol, in particular phloroglucinol, resorcinol, or aminophenol can then be added to the water phase and dissolved therein.

Alternatively, an oil phase can be produced in the method according to the invention by mixing a fragrance composition or a phase change material (PCM) with aromatic alcohols, in particular phloroglucinol, resorcinol or aminophenol. Alternatively, reactive monomers or diisocyanate derivatives can also be incorporated into the fragrance composition. Subsequently, the first temperature can be set.

A further step may be the preparation of a two-phase mixture by adding the oil phase to the water phase and subsequently increasing the rotational speed.

Subsequently, the emulsification can be started by adding formic acid. Regular determination of the particle size is possible here. Once the desired particle size is reached, the two-phase mixture can be stirred further and a second temperature for curing the capsule walls can be set. The second temperature may be in the range of from 55° C. to 65° C.

Subsequently, a melamine dispersion can be added to the microcapsule dispersion and a third temperature set, the third temperature preferably being in the range of from 75° C. to 85° C.

Another suitable step is the addition of an aqueous urea solution to the microcapsule dispersion. Subsequently, the emulsion stabilizer is added to the microcapsule dispersion before said dispersion is added to a solution of gelatin and alginate in order to produce the stabilization layer.

In this case, cooling to 45° C. to 55° C. would be carried out afterwards, and the pH of the microcapsule dispersion would be set to a value in the range of from 3.5 to 4.1, in particular 3.7.

The microcapsule dispersion can then be cooled to a fourth temperature, the fourth temperature being in the range of from 20° C. to 30° C. It can subsequently be cooled to a fifth temperature, the fifth temperature being in a range of from 4° C. to 17° C., in particular 8° C.

Subsequently, the pH of the microcapsule dispersion would be set to a value in the range of from 4.3 to 5.1 and glutaraldehyde or glyoxal would be added. The reaction conditions, in particular the temperature and pH, can be selected differently depending on the crosslinker. A person skilled in the art can derive the appropriate conditions, for example, from the reactivity of the crosslinker. The crosslinking density of the first layer (stability layer) and thus, for example, the tightness and degradability of the microcapsule shell is influenced by the amount of glutaraldehyde or glyoxal added. Accordingly, a person skilled in the art can vary the amount in a targeted manner in order to adapt the property profile of the microcapsule. A melamine suspension consisting of melamine, formic acid, and water can be prepared in order to produce the additional third layer. The melamine suspension is added to the microcapsule dispersion. Finally, the pH of the microcapsule dispersion would be set to a value in the range of from 9 to 12, in particular 10 to 11.

In addition, the microcapsule dispersion can be heated to a temperature in the range of from 20° C. to 80° C. for the curing in step e). As shown in Example 8, this temperature has an influence on the color resistance of the microcapsules. The temperature may be 20° C., 25° C., 30° C., 35° C., 40° C., 45° C., 50° C., 55° C., 60° C., 65° C., 70° C., 75° C., or 80° C. Below a temperature of 20° C., no influence on the color fastness is to be expected. A temperature of above 80° C. could have a negative effect on the microcapsule properties. According to one embodiment, the temperature is in the range of from 30° C. to 60° C. According to a preferred embodiment, the temperature is in the range of from 35° C. to 50° C.

According to one embodiment, the microcapsule dispersion is kept at the heating temperature for a period of at least 5 minutes. The period may be 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 100 minutes, 110 minutes, or 120 minutes. According to one embodiment, the microcapsule dispersion is kept at the heating temperature for a period of at least 30 minutes. According to one embodiment, the microcapsule dispersion is kept at the heating temperature for a period of at least 60 minutes.

### Microcapsule Dispersion and Coloring

Microcapsules are generally present in the form of microcapsule dispersions. Despite the use of aromatic alcohol in the barrier layer of the microcapsule shell, the microcapsule dispersions with the microcapsules described herein have only a low degree of coloration.

To qualify the discoloration, the spectral locus in the L*a*b* color space was determined for the microcapsules according to the invention. The L*a*b* color model is standardized in EN ISO 11664-4 “Colorimetry—Part 4: CIE 1976 L*a*b* Colour space”. The L*a*b* color space (also: CIELAB, CIEL*a*b*, Lab colors) describes all perceivable colors. It uses a three-dimensional color space in which the lightness value L* is perpendicular to the color plane (a*,b*). The a-coordinate indicates the chromaticity and color intensity between green and red and the b-coordinate indicates the chromaticity and color intensity between blue and yellow. The greater the positive a- and b-values and the smaller the negative a- and b- values, the more intense the hue. If a=0 and b=0, an achromatic hue is present on the lightness axis. The properties of the L*a*b* color model include device independence and perceptuality, which means that colors are defined independently of the type of production or reproduction technique thereof as they are perceived by a normal observer in standard light conditions.

As shown in the examples, the microcapsule dispersions according to the invention have a spectral locus with an L* value of at least 50 in the L*a*b* color space. The L″value may, for example, be 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80. According to a preferred embodiment, the microcapsule dispersions according to the invention have a spectral locus with an L″ value of at least 50 in the L*a*b* color space. The spectral locus is particularly preferably at least 60.

In addition, the microcapsule dispersions produced using the preparation method according to the invention are particularly color-stable. As shown in the examples, the spectral locus of the microcapsule dispersion in the L*a*b* color space after storage has an L* value of at least 50. The L* value after storage may be, for example, 51, 52, 53, 54, 55, 55, 56, 57, 58, 59, 60, 61, 62, 63, 64, 65, 66, 67, 68, 69, 70, 71, 72, 73, 74, 75, 76, 77, 78, 79, or 80. According to a preferred embodiment, the microcapsule dispersions according to the invention have a spectral locus with an L* value of at least 60 in the L*a*b* color space after storage. The spectral locus is particularly preferably at least 65.

The microcapsule dispersion can contain a thickening agent, such as Jaguar HP105 (Solvay). The thickening agent serves in particular to adjust the viscosity. An increase in viscosity, for example up to a viscosity of 2500 mPas (measured using Brookfield, RT, S3), can stabilize the microcapsule dispersion and thus improve the stirrability and storage.

According to one embodiment, the storage time is at least four weeks, preferably at least six weeks, and in particular at least eight weeks.

### EXAMPLES

### Example 1—Preparation of a Reference Microcapsule—which is not in ACCORDANCE with the Invention—with Melamine-Formaldehyde Formulation

### 1.1 Materials

The materials used for preparing the melamine-formaldehyde reference microcapsules are shown in Table 1.

TABLE 1
List of substances used to prepare MC2

| | Substance | | MC2 |
|---|---|---|---|
| Trade name* | designation | Concentration/wt. % | Initial weight/g |
| — | DI water | 100 | 187.5 |
| Dimension ™ SD from | 1,3,5-Triazine-2,4,6- | 67 | 42.5* |
| Solenis | triamine, polymer with | | |
| | formaldehyde, | | |
| | methylated (content | | |
| | (W/W): >=60%-<=80%), | | |
| | in water | | |
| Dimension ™ PA 140 | Polymer based on: | 20 | 35.0* |
| from Solenis | acrylamidosulfonate | | |
| Weiroclean from Kurt | Core material (e.g. | 100 | 192.5* |
| Kitzing GmbH | fragrance oil, PCM, | | |
| | etc.) | | |
| — | Formic acid | 10 | 8.8 |
| Melafine ® from OCI | Melamine suspension ¹⁾ | 27 | 48.8 |
| Nitrogen B.V. | | | |
| — | Urea solution | 28.6 | 70.0 |

¹⁾ Concentration based on the acidified suspension
*Amounts of the components refer to the commercial goods and are used as supplied.

### 1.2 Preparation Method (Based on BASF Patent EP 1 246693 B1)

Dimension SD was stirred into DI water and then Dimension PA140 was added and stirred until a clear solution was formed. The solution was heated to 30-35° C. in a water bath. During stirring with a dissolver disk, the perfume oil was added at 1100 rpm. The PH of the oil-in-water emulsion was set to 3.3-3.8 using 10% formic acid. Subsequently, the emulsion was stirred further at 1100 rpm for 30 min until a droplet size of 20-30 μm was reached or longer until the desired particle size of 20-30 μm (peak max) is reached. The particle size was determined by means of a Beckman Coulter device (laser diffraction, Fraunhofer method). The speed was reduced as a function of the viscosity in order to ensure good mixing. Stirring took place at this speed for a further 30 min at 30 to 40° C. Subsequently, the emulsion was heated to 60° C. and stirred further. The melamine suspension was set to a pH of 4.5 using formic acid (10%) and metered into the reaction mixture. The batch was kept at 60° C. for 60 min and then heated to 80° C. After stirring at 80° C. for 60 min, the urea solution was added.

After cooling to room temperature, the microcapsule dispersion was filtered through a 200 μm mesh filter.

### 1.3 Result

The MF reference microcapsule MC2 obtained was examined under a light microscope.

A typical recording of the MC2 is shown in FIG. 1F. To evaluate the microcapsules obtained, the pH, the solids content, the viscosity, the particle size, the content of core material in the slurry, and also the L* value of the spectral locus were determined. The result is shown in Table 2.

TABLE 2
Analysis results of the reference microcapsule
MC2 not according to the invention

| | Measurement method | MC2 |
|---|---|---|
| pH | pH electrode | 5.5-6.5 |
| Solids content/% | Microwave | 37-41 |
| Viscosity/mPas | Brookfield viscometer | <1000 |
| Particle size peak max./μm | Laser diffraction | 20-30 |
| Core material | Calculation from formulation | 30-35 |
| content in slurry/% | | |
| L* value/— | Spectrophotometer | 65-80 |

### Example 2—Preparation of Slurry 2 and Slurry 5 Microcapsule Dispersions According to the Invention and of MC1 Reference Capsule not According to the Invention

### 2.1 Materials

The materials used for preparing slurry 2 and slurry 5 microcapsules according to the invention are shown in Table 3.

TABLE 3
List of substances used to prepare slurry 2 and 5

| | | | Slurry | Slurry |
|---|---|---|---|---|
| | | | 2 | 5 |
| | | Concent | Initial | Initial |
| | | ration/ | weight/ | weight/ |
| Trade name | Substance designation | wt. % | g | g |
| | DI water addition 1 | 100 | 35.2 | 39.6 |
| Dimension ™ SD | 1,3,5-Triazine- | 67 | 2.1* | 1.7* |
| from Solenis | 2,4,6-triamine, | | | |
| | polymer with | | | |
| | formaldehyde, | | | |
| | methylated (content | | | |
| | (W/W): | | | |
| | >=60%-<=80%), | | | |
| | in water | | | |
| Dimension ™ PA | Polymer based on: | 20 | 4.6* | 3.8* |
| 140 from Solenis | acrylamidosulfonate | | | |
| | addition 1 | | | |
| Weiroclean from | Core material (e.g. | 100 | 52.8* | 52.6* |
| Kurt Kitzing GmbH | fragrance oil, PCM, etc.) | | | |
| — | Resorcinol solution | 3.2 | 12.7 | 11.9 |
| — | Formic acid addition 1 | 10 | 0.7 | 0.4 |
| Melafine ® from | Melamine suspension | 27 | 2.6 | 2.0 |
| OCI Nitrogen B.V. | addition 1 ¹⁾ | | | |
| — | Urea solution | 41.9 | 2.6 | 2.2 |
| Dimension ™ PA | Polymer based on: | 20 | 9.5* | 9.6* |
| 140 from Solenis | acrylamidosulfonate | | | |
| | addition 2 | | | |
| — | Tap water | 100 | 136.2 | 135.6 |
| — | Sodium sulfate | 100 | 0.6* | 0.6* |
| Scogin ® MV from | Sodium alginate | 100 | 2.0* | 2.0* |
| DuPont Nutrition | | | | |
| Ireland | | | | |
| Edible gelatin | Porcine gelatin | 100 | 8.4* | 8.4* |
| powder from Ewald- | | | | |
| Gelatine GmbH | | | | |
| — | Formic acid addition 2 | 20 | 2.5 | 2.5 |
| — | Sodium hydroxide | 20 | 2.4 | 2.3 |
| | addition 1 | | | |
| Glutaraldehyde, | Glutaraldehyde solution | 50 | 2.6* | 2.6* |
| 50% aq. soln. from | | | | |
| Alfa Aesar | | | | |
| Melafine ® from | Melamine suspension | 27 | 27.5 | 27.4 |
| OCI Nitrogen B.V. | addition 2 ¹⁾ | | | |
| — | Sodium hydroxide | 20 | 4.7 | 4.7 |
| | addition 2 | | | |

¹⁾ Concentration based on the acidified suspension.
2) The given amounts for acids/alkalis are guidelines. They are adjusted to the pH range mentioned in the experimental procedure.
*Amounts of the components refer to the commercial goods and are used as supplied.

The materials used for preparing the MC1 reference microcapsules are shown in Table 4.

TABLE 4
List of substances used to prepare MC1

| | | MC1 | |
|---|---|---|---|
| | | Concentration/ | Initial weight/ |
| Trade name | Substance designation | wt. % | g |
| | DI water addition 1 | 100 | 34.9 |
| Dimension ™ SD from | 1,3,5-Triazine-2,4,6-triamine, | 67 | 1.6* |
| Solenis | polymer with formaldehyde, | | |
| | methylated (content | | |
| | (W/W): >=60%-<=80%), in water | | |
| Dimension ™ PA 140 | Polymer based on: | 20 | 3.4* |
| from Solenis | acrylamidosulfonate | | |
| Weiroclean from Kurt | Core material (e.g. fragrance oil, | 100 | 38.8* |
| Kitzing GmbH | PCM, etc.) | | |
| — | Resorcinol solution | 12.2 | 2.5 |
| — | Formic acid addition 1 | 20 | 0.5 |
| Melafine ® from OCI | Melamine suspension addition 1 ¹⁾ | 27 | 1.9 |
| Nitrogen B.V. | | | |
| — | Urea solution | 16.6 | 4.7 |
| — | Tap water | 100 | 100.19 |
| — | Sodium sulfate | 100 | 0.5* |
| Edible gelatin powder | Porcine gelatin | 100 | 6.2* |
| from Ewald-Gelatine | | | |
| GmbH | | | |
| Scogin ® MV from | Sodium alginate | 100 | 1.4* |
| DuPont Nutrition | | | |
| Ireland | | | |
| — | Formic acid addition 2 | 20 | 1.4 |
| — | Sodium hydroxide addition 1 | 20 | 0.8 |
| — | Glutaraldehyde solution | 50 | 1.9 |
| Melafine ® from OCI | Melamine suspension addition 2 ¹⁾ | 27 | 6.7 |
| Nitrogen B.V. | | | |
| — | Sodium hydroxide addition 2 | 20 | 2.2 |

¹⁾ Concentration based on the acidified suspension.
2) The given amounts for acids/alkalis are guidelines. They are adjusted to the pH range mentioned in the experimental procedure.
*Amounts of the components refer to the commercial goods and are used as supplied.

2.2 Preparation Method for the MC1 Microcapsules not ACCORDING to the invention

To prepare the reaction mixture 1, Dimension PA140 and Dimension SD were weighed with DI water addition 1 in a beaker and premixed using a 4 cm dissolver disk. The beaker was fixed in the water bath and stirred with the dissolver disc at 500 rpm and 30° C. until a clear solution formed.

As soon as the Dimension SD/Dimension PA140 solution was clear and reached 30-40° C., the amount of perfume oil was slowly added and the speed was adjusted in such a way (1100 rpm) that the desired particle size is thus achieved. The pH of this mixture was then acidified by adding formic acid addition 1. It was emulsified for 20-30 min or for longer until the desired particle size of 20-30 μm (peak max) is reached. The particle size was determined by means of a Beckman Coulter device (laser diffraction, Fraunhofer method). After the particle size was reached, the speed was reduced in order to ensure gentle mixing.

Subsequently, the resorcinol solution was stirred in and preformed under gentle stirring for 30-40 min. After the preforming time had elapsed, the emulsion temperature was increased to 50° C. within 15 min. Upon reaching this temperature, the mixture was increased to 60° C. over a period of 15 min and this temperature was maintained for a further 30 min. Subsequently, the melamine suspension was adjusted to a pH of 4.5 using 20% formic acid and metered into the reaction mixture over a period of 90 min. Afterwards, the temperature was maintained for 30 min. After the 30 min had elapsed, the temperature was initially increased to 70° C. within 15 min. The temperature was then increased to 80° C. within 15 minutes and maintained for 120 minutes. Afterwards, the aqueous urea solution was added, the heat source was switched off, and the reaction mixture 1 was cooled to room temperature. In a separate beaker, sodium sulfate was dissolved in tap water while being stirred using a paddle stirrer at 40-50° C. Sodium alginate and porcine gelatin are slowly sprinkled into the heated water. After all solids had been dissolved, reaction mixture 1 was added to the prepared gelatin/sodium alginate solution while being stirred. When a homogeneous mixture was obtained, the pH value was adjusted to 3.9 by slow dropwise addition of the formic acid addition 2, then the heat source was removed. Subsequently, the batch was cooled to room temperature. After reaching room temperature, the reaction mixture was cooled using ice. When a temperature of 8° C. was reached, the ice bath was removed and the pH was increased to 4.7 with sodium hydroxide addition 1. Glutaraldehyde was then added. Care was taken to ensure that the temperature does not exceed 16-20° C. until the glutaraldehyde is added.

Subsequently, the melamine suspension addition 2 acidified by means of 20% formic acid to a pH of 4.5 was metered in slowly. Then, the reaction mixture was heated to 60° C. and, when reached, said temperature was maintained for 60 min. After this holding time, the heat source was removed and the microcapsule suspension was gently stirred for 14 hours. After 14 hours, the microcapsule suspension was adjusted to a pH of 10.5 by adding Sodium hydroxide solution addition 2.

### 2.2.1 Result

The resulting microcapsule MC1 was examined under a light microscope. Typical recordings are shown in FIG. 1E. To evaluate MC1, the pH, the solids content, the viscosity, the particle size, the content of core material in the slurry, and also the L* value of the spectral locus were determined. The result is shown in Table 5.

TABLE 5
Analysis results of the reference microcapsule
MC1 not according to the invention

| | Measurement method | MC1 |
|---|---|---|
| pH | pH electrode | 8-10 |
| Solids content/% | Microwave | 20-30 |
| Viscosity/mPas | Brookfield viscometer | <1000 |
| Particle size peak max./μm | Laser diffraction | 20-30 |
| Core material | Calculation from formulation | 15-20 |
| content in slurry/% | | |
| L* value/— | Spectrophotometer | 55-75 |

2.3 Preparation Method for the Slurry 2 and Slurry 5 Microcapsule Dispersions According to the invention

To prepare the reaction mixture 1, Dimension PA140 addition 1 and Dimension SD were weighed with DI water addition 1 in a beaker and premixed using a 4 cm dissolver disk. The beaker was fixed in the water bath and stirred with the dissolver disc at 500 rpm and 30° C. until a clear solution formed.

As soon as the Dimension SD/Dimension PA140 solution was clear and reached 30-40° C., the core material was slowly added and the speed was adjusted in such a way (e.g. 1100 rpm) that the desired particle size is thus achieved. The pH of this mixture was then acidified by adding formic acid addition 1 (pH=3.3-3.5).

It was emulsified for 20-30 min or for longer until the desired particle size of 20-30 μm (peak max) is reached. The particle size was determined by means of a Beckman Coulter device (laser diffraction, Fraunhofer method). After the particle size was reached, the speed was reduced in order to ensure gentle mixing and the resorcinol solution was added.

Gentle stirring was performed for 30-40 min. After the preforming time had elapsed, the emulsion temperature was increased to 50° C. within 15 min. Upon reaching this temperature, the mixture was increased to 60° C. over a period of 15 min and this temperature was maintained for a further 30 min. The Melafin suspension addition 1 was then adjusted to a pH of 4.5 with the aid of 20% formic acid and metered into the reaction mixture over a period of 90 minutes.

Afterwards, the temperature was maintained for 30 min. After the 30 min had elapsed, the temperature was initially increased to 70° C. within 15 min. Subsequently, the temperature was increased to 80° C. within 15 min and maintained for 90 min.

Afterwards, the aqueous urea solution was added, the heat source was switched off, and the reaction mixture 1 was cooled to room temperature. After reaction mixture 1 has reached room temperature, Dimension PA140 addition 2 is added.

In a separate beaker, sodium sulfate was dissolved in tap water while being stirred using a paddle stirrer at 40-50° C. Sodium alginate and porcine gelatin are slowly sprinkled into the heated tap water. After all solids had been dissolved, reaction mixture 1 was added to the prepared gelatin/sodium alginate solution while being stirred. When a homogeneous mixture was obtained, the pH value was adjusted to 3.7 by slow dropwise addition of the formic acid addition 2, then the heat source was removed and the batch was cooled naturally to room temperature.

After reaching room temperature, the reaction mixture was cooled using ice. When a temperature of 8° C. was reached, the ice bath was removed and the pH was increased to 4.7 with sodium hydroxide addition 1. Glutaraldehyde 50% was then added. Care was taken to ensure that the temperature does not exceed 16-20° C. until the glutaraldehyde 50% is added.

Subsequently, the melamine suspension addition 2 acidified by means of 20% formic acid to a pH of 4.5 was metered in within a period of approx. 2 min. The microcapsule dispersion was subsequently stirred gently at room temperature for 14 h. After the 14 h had elapsed, the microcapsule dispersion was adjusted to a pH of 10.5 within a period of approx. 15 min by means of sodium hydroxide addition 2.

### 2.3.1 Result

The obtained slurry 2 and slurry 5 microcapsules according to the invention were examined by light microscopy. Typical recordings are shown in FIGS. 1A and 1C. To evaluate slurry 2 and slurry 5, the pH, the solids content, the viscosity, the particle size, the content of core material in the slurry, and also the L* value of the spectral locus were determined. The result is shown in Table 6.

TABLE 6
Analysis results of the slurry 2 and slurry 5 microcapsule
dispersions according to the invention

| | Measurement method | Slurry 2 and slurry 5 |
|---|---|---|
| pH | pH electrode | 8-10 |
| Solids content/% | Microwave | 20-30 |
| Viscosity/mPas | Brookfield viscometer | <1000 |
| Particle size peak max./μm | Laser diffraction | 20-30 |
| Core material content in slurry/% | Calculation from formulation | 15-20 |
| L* value/— | Spectrophotometer | 61-67 |

### Example 3—Preparation of Slurry 3 and Slurry 6 Microcapsule Dispersions According to the Invention and of MC4 Reference Microcapsule Dispersion not accoRding to the Invention 3.1 Materials

The materials used for preparing slurry 3 and slurry 6 microcapsules according to the invention are shown in Table 7.

TABLE 7
List of substances used to prepare slurry 3 and 6

| | Substance | | Slurry 3 | Slurry 6 |
|---|---|---|---|---|
| Trade name | designation | Concentration/wt. % | Initial weight/g | Initial weight/g |
| — | DI water addition 1 | 100 | 35.2 | 39.6 |
| Dimension ™ | 1,3,5-triazine-2,4,6- | 67 | 2.1* | 1.7* |
| SD from | triamine, polymer | | | |
| Solenis | with formaldehyde, | | | |
| | methylated, | | | |
| | aqueous | | | |
| Dimension ™ | Polymer based on: | 20 | 4.6* | 3.8* |
| PA 140 from | acrylamidosulfonate | | | |
| Solenis | addition 1 | | | |
| Weiroclean | Core material (e.g. | 100 | 52.8* | 52.6* |
| from Kurt | fragrance oil, PCM, | | | |
| Kitzing | etc.) | | | |
| GmbH | | | | |
| — | Resorcinol solution | 3.2 | 12.7 | 11.9 |
| — | Formic acid | 10 | 0.7 | 0.4 |
| | addition 1 | | | |
| Melafine ® | Melamine | 27 | 2.6 | 2.0 |
| from OCI | suspension | | | |
| Nitrogen | (pH = 4.5) addition 1 ¹⁾ | | | |
| B.V. | | | | |
| — | Urea solution | 41.9 | 2.6 | 2.2 |
| Dimension ™ | Polymer based on: | 20 | 9.5* | 9.6* |
| PA 140 from | acrylamidosulfonate | | | |
| Solenis | addition 2 | | | |
| — | Tap water | 100 | 136.2 | 135.6 |
| — | Sodium sulfate | 100 | 0.6* | 0.6* |
| Scogin ® MV | Sodium alginate | 100 | 2.0* | 2.0* |
| from DuPont | | | | |
| Nutrition | | | | |
| Ireland | | | | |
| Edible | Porcine gelatin | 100 | 8.4* | 8.4* |
| gelatin | | | | |
| powder from | | | | |
| Ewald- | | | | |
| Gelatine | | | | |
| GmbH | | | | |
| — | Formic acid | 20 | 2.5 | 2.5 |
| | addition 2 | | | |
| — | Sodium hydroxide | 20 | 2.4 | 2.3 |
| | addition 1 | | | |
| Glyoxal 40% | Glyoxal | 40 | 1.9* | 1.9* |
| solution in | | | | |
| water for | | | | |
| synthesis, | | | | |
| merck KGaA | | | | |
| Melafine ® | Melamine | 27 | 27.5 | 27.4 |
| from OCI | suspension | | | |
| Nitrogen | (pH = 4.5) addition 2 ¹⁾ | | | |
| B.V. | | | | |
| — | Sodium hydroxide | 20 | 4.7 | 4.7 |
| | addition 2²⁾ | | | |

¹⁾ Concentration based on the acidified suspension
²⁾The given amounts for acids/alkalis are guidelines. They are adjusted to the pH range mentioned in the experimental procedure.
*Amounts of the components refer to the commercial goods and are used as supplied.

The materials used for preparing the MC4 reference microcapsules are shown in Table 8.

TABLE 8
List of substances used to prepare MC4

| Trade name | Substance designation | Concentration/wt. % | Initial weight/g |
|---|---|---|---|
| — | Water addition 1 | 100 | 34.9 |
| Dimension ™ SD | 1,3,5-triazine-2,4,6- | 67 | 1.6* |
| from Solenis | triamine, polymer with | | |
| | formaldehyde, | | |
| | methylated, aqueous | | |
| Dimension ™ PA 140 | Polymer based on: | 20 | 3.4* |
| from Solenis | acrylamidosulfonate | | |
| | addition 1 | | |
| Weiroclean from Kurt | Core material (e.g. | 100 | 38.8* |
| Kitzing GmbH | fragrance oil, PCM, etc.) | | |
| — | Resorcinol solution | 12.2 | 2.5 |
| — | Formic acid addition 1 | 20 | 0.5 |
| Melafine ® from OCI | Melamine suspension | 27 | 1.9 |
| Nitrogen B.V. | (pH = 4.5) Addition 1 ¹⁾ | | |
| — | Urea solution | 16.6 | 4.7 |
| — | Tap water | 100 | 100.2 |
| — | Sodium sulfate | 100 | 0.5* |
| Scogin ® MV from | Sodium alginate | 100 | 1.4* |
| DuPont Nutrition | | | |
| Ireland | | | |
| Edible gelatin | Porcine gelatin | 100 | 6.2* |
| powder from Ewald- | | | |
| Gelatine GmbH | | | |
| — | Formic acid* addition 2²⁾ | 20 | 1.4 |
| — | Sodium hydroxide | 20 | 0.8 |
| | addition 1²⁾ | | |
| — | Glyoxal | 40 | 1.4 |
| Melafine ® from OCI | Melamine suspension | 27 | 6.7 |
| Nitrogen B.V. | (pH = 4.5) Addition 2 ¹⁾ | | |
| — | Sodium hydroxide | 20 | 2.2 |
| | addition 2²⁾ | | |

¹⁾ Concentration based on the acidified suspension
²⁾The given amounts for acids/alkalis are guidelines. They are adjusted to the pH range mentioned in the experimental procedure.
*Quantities of the components refer to the merchandise and are used as supplied

### 3.2. Preparation Method for the MC4 Microcapsules not According to the Invention

To prepare the reaction mixture 1, Dimension PA140 and Dimension SD were weighed with water addition 1 in a beaker and premixed using a 4 cm dissolver disk. The beaker was fixed in the water bath and stirred with the dissolver disc at 500 rpm and 30° C. until a clear solution formed.

As soon as the Dimension SD/Dimension PA140 solution was clear and reached 30-40° C., the core material was slowly added and the speed was adjusted in such a way (e.g. 1100 rpm) that the desired particle size is thus achieved. The pH of this mixture was then acidified by adding formic acid addition 1. It was emulsified for 20-30 min or for longer until the desired particle size of 20-30 μm (peak max) is reached. The particle size was determined by means of a Beckman Coulter device (laser diffraction, Fraunhofer method). After the particle size was reached, the speed was reduced in order to ensure gentle mixing.

Subsequently, the resorcinol solution was stirred in and preformed under gentle stirring for 30-40 min. After the preforming time had elapsed, the emulsion temperature was increased to 50° C. within 15 min. Upon reaching this temperature, the mixture was increased to 60° C. over a period of 15 min and this temperature was maintained for a further 30 min. Subsequently, the melamine suspension was adjusted to a pH of 4.5 using 20% formic acid and metered into the reaction mixture over a period of 90 min. Afterwards, the temperature was maintained for 30 min. After the 30 min had elapsed, the temperature was initially increased to 70° C. within 15 min. The temperature was then increased to 80° C. within 15 minutes and maintained for 120 minutes. Afterwards, the aqueous urea solution was added, the heat source was switched off, and the reaction mixture 1 was cooled to room temperature. In a separate beaker, sodium sulfate was dissolved in tap water while being stirred using a paddle stirrer at 40-50° C. Sodium alginate and porcine gelatin are slowly sprinkled into the heated water. After all solids had been dissolved, reaction mixture 1 was added to the prepared gelatin/sodium alginate solution while being stirred. When a homogeneous mixture was obtained, the pH value was adjusted to 3.9 by slow dropwise addition of the formic acid addition 2, then the heat source was removed. Subsequently, the batch was cooled to room temperature. After reaching room temperature, the reaction mixture was cooled using ice. When a temperature of 8° C. was reached, the ice bath was removed and the pH was increased to 4.7 with sodium hydroxide addition 1. Then the glyoxal solution was added. Care was taken to ensure that the temperature before the glyoxal solution was added did not exceed 16-20° C. Subsequently, the melamine suspension addition 2 acidified by means of 20% formic acid to a pH of 4.5 was metered in slowly. Then, the reaction mixture was heated to 60° C. and, when reached, said temperature was maintained for 60 min. After this holding time, the heat source was removed and the microcapsule suspension was gently stirred for 14 hours. After 14 hours, the microcapsule suspension was adjusted to a pH of 10.5 by adding Sodium hydroxide solution addition 2.

### 3.2.1 Result

The resulting microcapsule MC4 was examined under a light microscope. Typical recordings are shown in FIG. 1G. To evaluate MC1, the pH, the solids content, the viscosity, the particle size, the content of core material in the slurry, and also the L* value of the spectral locus were determined. The result is shown in Table 9.

TABLE 9
Analysis results of the reference microcapsule
MC4 not according to the invention

| | Measurement method | MC4 |
|---|---|---|
| pH | pH electrode | 8-10 |
| Solids content/% | Microwave | 20-30 |
| Viscosity/mPas | Brookfield viscometer | <1000 |
| Particle size peak max./μm | Laser diffraction | 20-30 |
| Core material | Calculation from formulation | 15-20 |
| content in slurry/% | | |
| L* value/— | Spectrophotometer | 51-54 |

### 3.3. Preparation Method for Slurry 3 and Slurry 6 Microcapsule Dispersions According to the Invention

To prepare the reaction mixture 1, Dimension PA140 addition 1 and Dimension SD were weighed with DI water addition 1 in a beaker and premixed using a 4 cm dissolver disk. The beaker was fixed in the water bath and stirred with the dissolver disc at 500 rpm and 30° C. until a clear solution formed.

As soon as the Dimension SD/Dimension PA140 solution was clear and reached 30-40° C., the core material was slowly added and the speed was adjusted in such a way (e.g. 1100 rpm) that the desired particle size is thus achieved. The pH of this mixture was then acidified by adding formic acid addition 1 (pH=3.3-3.5).

It was emulsified for 20-30 min or for longer until the desired particle size of 20-30 μm (peak max) is reached. The particle size was determined by means of a Beckman Coulter device (laser diffraction, Fraunhofer method). After the particle size was reached, the speed was reduced in order to ensure gentle mixing and the resorcinol solution was then added.

Gentle stirring was performed for 30-40 min. After the preforming time had elapsed, the emulsion temperature was increased to 50° C. within 15 min. Upon reaching this temperature, the mixture was increased to 60° C. over a period of 15 min and this temperature was maintained for a further 30 min. Subsequently, the melamine suspension was adjusted to a pH of 4.5 using 20% formic acid and metered into the reaction mixture over a period of 90 min.

Afterwards, the temperature was maintained for 30 min. After the 30 min had elapsed, the temperature was initially increased to 70° C. within 15 min. Subsequently, the temperature was increased to 80° C. within 15 min and maintained for 90 min.

Afterwards, the aqueous urea solution was added, the heat source was switched off, and the reaction mixture 1 was cooled to room temperature. After reaction mixture 1 has reached room temperature, Dimension PA140 addition 2 is added.

In a separate beaker, sodium sulfate was dissolved in tap water while being stirred using a paddle stirrer at 40-50° C. Sodium alginate and porcine gelatin are slowly sprinkled into the heated tap water. After all solids had been dissolved, reaction mixture 1 was added to the prepared gelatin/sodium alginate solution while being stirred. When a homogeneous mixture was obtained, the pH value was adjusted to 3.7 by slow dropwise addition of the formic acid addition 2, then the heat source was removed and the batch was cooled naturally to room temperature.

After reaching room temperature, the reaction mixture was cooled with ice to a temperature of 8° C. and the temperature was maintained at 8° C. The pH was increased to 4.7 by sodium hydroxide solution addition 1. Subsequently, glyoxal 40% was added at a temperature of 8° C. and the melamine suspension addition 2 acidified by 20% formic acid to a pH of 4.5 was subsequently metered in within a period of about 2 min. By means of sodium hydroxide addition 2, the pH was adjusted to a value of pH=10.5 within a period of about 15 min. The ice bath is removed and the reaction mixture is heated to 40° C. and kept at this temperature for 1 h.

After this holding time, the microcapsule suspension was stirred gently for 14 h at room temperature.

### 3.3.1 Result

The obtained microcapsules slurry 3 and slurry 6 according to the invention were examined by light microscopy. Typical recordings are shown in FIGS. 1B and D. To evaluate slurry 3 and slurry 6, the pH, the solids content, the viscosity, the particle size, the content of core material in the slurry, and also the L′ value of the spectral locus were determined. The result is shown in Table 10.

TABLE 10
Analysis results of the microcapsule dispersion
slurry 3 and slurry 6 according to the invention

| | Measurement method | Slurry 3 and slurry 6 |
|---|---|---|
| pH | pH electrode | 6-10 |
| Solids content/% | Microwave | 20-30 |
| Viscosity/mPas | Brookfield viscometer | <1000 |
| Particle size peak max./μm | Laser diffraction | 20-30 |
| Core material content in slurry/% | Calculation from formulation | 15-20 |
| L* value/— | Spectrophotometer | 55-59 |

### Example 4—Microscopic Determination of Stability Layer Thickness

### 4.1 Preliminary Remarks

The layer thickness of the stability layer can be determined in principle in two ways. Firstly, there is the light microscopic approach, i.e. the direct optical measurement of the observed layer thickness by means of a microscope and corresponding software.

A second possibility is the measurement of the particle size distribution by means of laser diffraction. Here, the modal value of a particle size distribution of the pure barrier template (cf. Example 1) can be set in comparison with the modal value of a particle size distribution for a microcapsule according to the invention. The increase in this measured value should reflect the increase in the hydrodynamic diameter (due to the application of the stability layer) of the main fraction on measured microcapsules. The formation of the difference from the two measured modal values ultimately results in twice the layer thickness of the stability layer.

Both measurement methods yielded corresponding measurement results in preliminary tests, for which reason only the performance and the result of the light microscopic examination have been reproduced in the following.

### 4.2 Experimental Procedure

The layer thickness of the stability layer was determined by light microscopy on an Olympus BX50 microscope. The software OLYMPUS Stream Essentials 2.4.2 (Build 20105) was used for the measurement.

Initially, a highly diluted sample of the capsule slurries according to the invention was produced with tap water. A drop of this dispersion was applied to a microscope slide and provided with a cover glass.

To determine the layer thickness, a magnification of 500x was selected on the microscope and the corresponding microcapsules according to the invention were focused in the applied sample dispersion.

In the above software, the diameter of the visible barrier plate was subsequently detected with the function “3-point circle”. By means of the ruler function, the layer thickness of the stability layer could ultimately be measured at three characteristic points.

Due to the elliptical shape of the stability layer, a plurality of layer thickness measurement values were recorded for each focused microcapsule in order to reproduce the variance in the layer thickness. The microcapsules according to the invention have a greater layer thickness at two opposite apexes and a smaller layer thickness at the other two opposite apexes. To reduce this measurement error in the specification of a layer thickness for the stability layer, an average value over 10 individual microcapsules was prepared.

### 4.3 Results

TABLE 11
Results of the layer thickness measurement of the
stabilization layer using the example of slurry 3

| | | Layer thickness/μm | | |
|---|---|---|---|---|
| No. considered capsule/— | Barrier template diameter/μm | M1 | M2 | M3 |
| 1 | 30.48 | 3.02 | 7.86 | 4.02 |
| 2 | 26.12 | 4.74 | 3.37 | 4.36 |
| 3 | 25.78 | 4.76 | 4.25 | 3.21 |
| 4 | 25.81 | 5.74 | 4.53 | 2.23 |
| 5 | 24.28 | 8.12 | 2.38 | 7.17 |
| 6 | 16.08 | 2.28 | 3.72 | 1.73 |
| 7 | 30.86 | 3.75 | 3.21 | 5.56 |
| 8 | 20.37 | 2.03 | 3.18 | 4.01 |
| 9 | 36.83 | 3.23 | 4.68 | 3.21 |
| 10 | 20.74 | 3.86 | 2.04 | 3.54 |

| | |
|---|---|
| Average | 3.99 |
| SD | 1.59 |

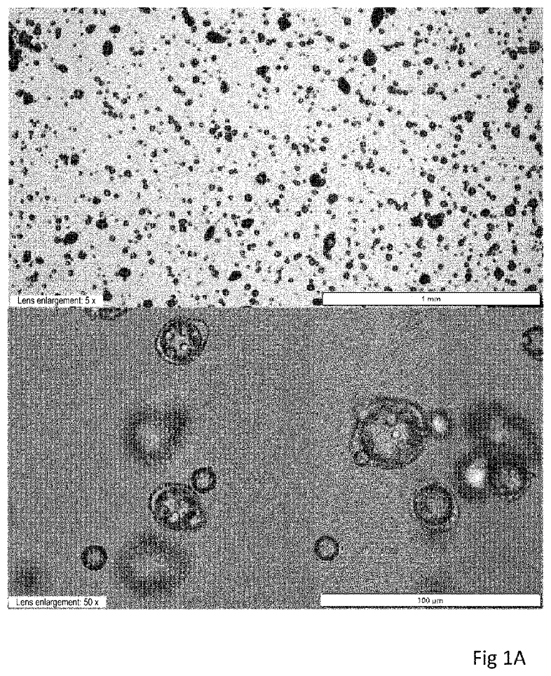

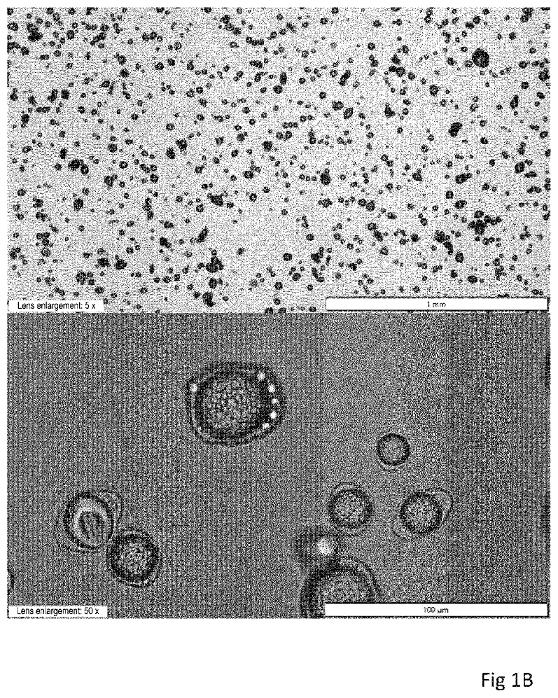

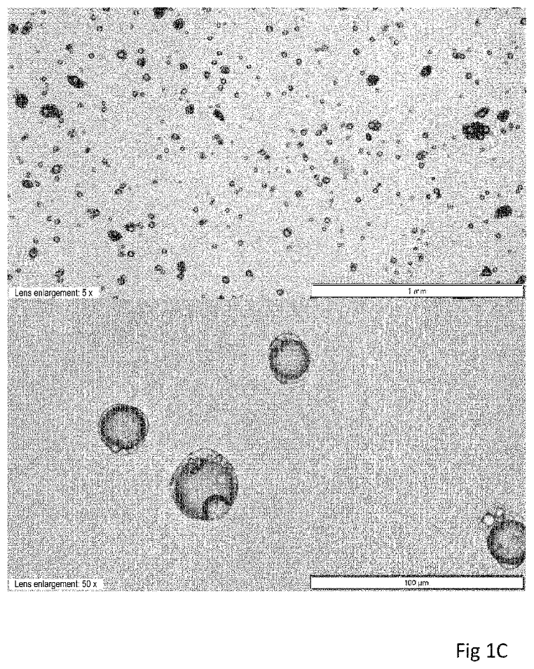

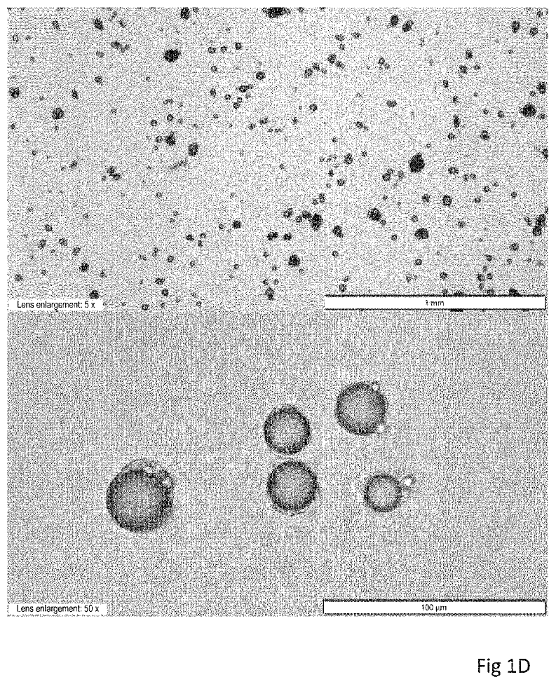

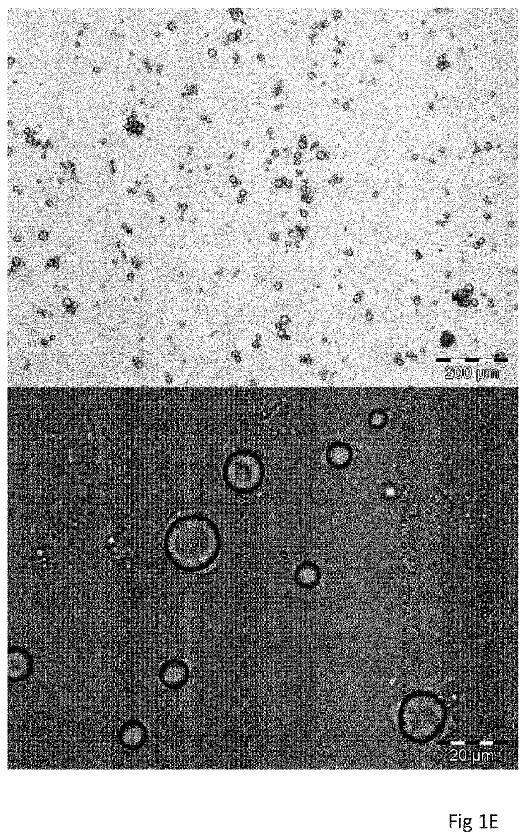

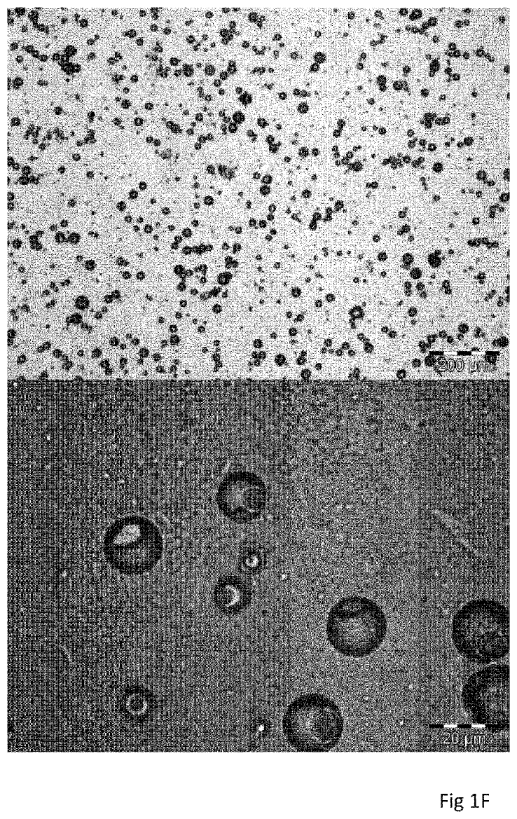

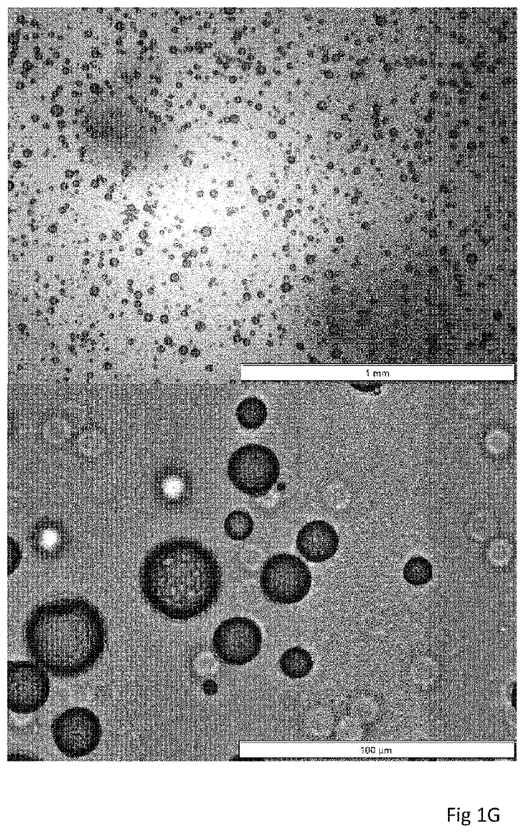

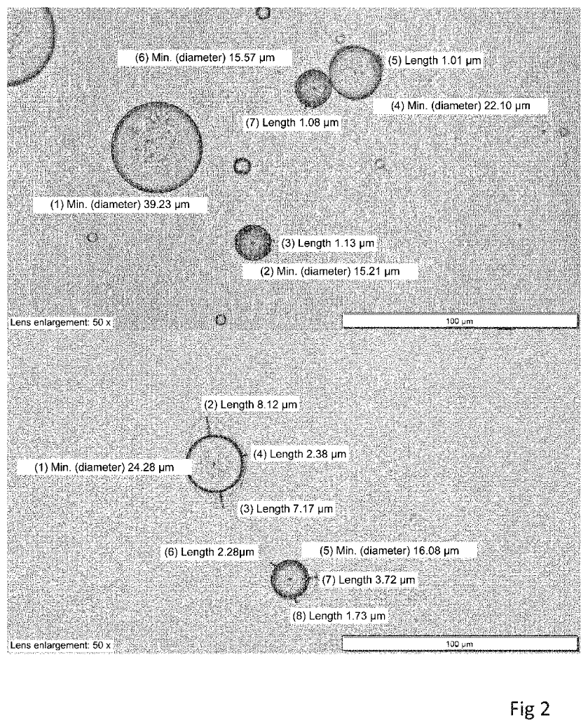

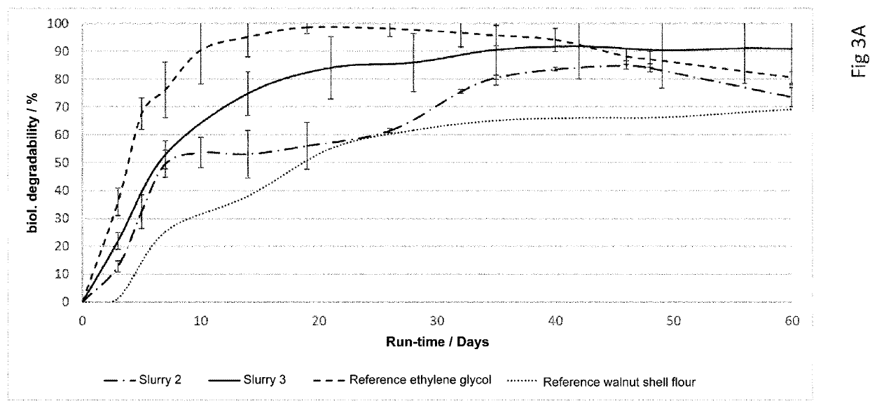

By way of example, the light microscope images of the produced layer thickness measurement for slurry 3 and a comparison of the microcapsule MC1 are shown in FIG. 2. The result of the measurement of 10 capsules of the slurry 3 is shown in Table 11.

This result was confirmed for slurry 3 by means of laser diffraction and via the difference between the modal value of the particle size distribution and a microcapsule without the stability layer.

### Example 5—Stability Measurement of the Microcapsules

### 5.1 Preliminary Remark

To determine the stability of microcapsules, they were stored in a model fabric softener formulation at 40° C. for a period of up to 4 weeks and the concentration of the fragrances diffused from the interior of the capsule into the surrounding formulation was determined using HS-GC/MS. Based on the measured values, the residual proportion of the perfume oil still in the capsule was calculated.

Model fabric softener formulation based on Rewoquat WE 18 E US from Evonik based on the recipe from the associated product data sheet:

To produce the fabric softener base, 94 g of water were heated to 50° C. and 5.65 g of Rewoquat WE 18 E US were added to the heated water with stirring. The mixture was cooled to room temperature, then the microcapsule dispersion was added.

### 5.2 Experimental Procedure

For this purpose, the slurry 2, slurry 3, slurry 5, slurry 6 and MC1, MC2, and MC4 microcapsule dispersions were carefully homogenized and stored at a concentration of 1 wt. % in the model formulation at 40° C., sealed airtight, in the heating cabinet. The non-encapsulated fragrance with an analogous concentration of fragrance in the model formulation serves as a comparison.

After a specified storage period, the samples were removed from the heating cabinet and an aliquot was weighed into a 20 ml headspace vial. The vial was then immediately sealed.

These samples were prepared by Headspace SPME (solid-phase microextraction) with the aid of capillary gas chromatography and evaluated, after detection with a mass-selective detector (MSD).

### 5.3 Result

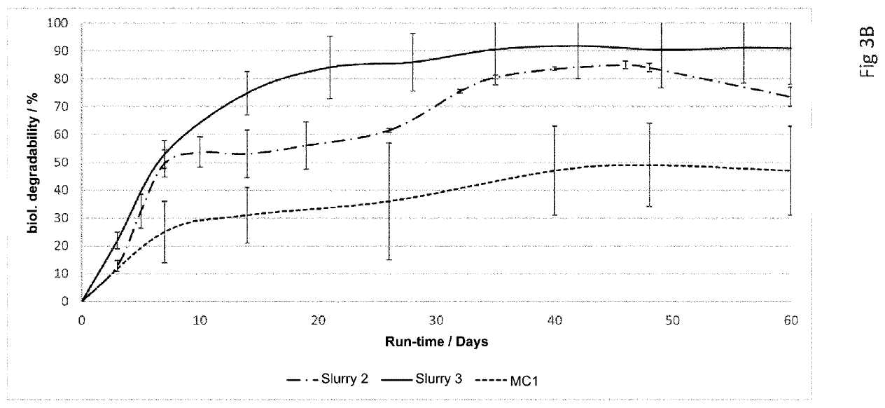

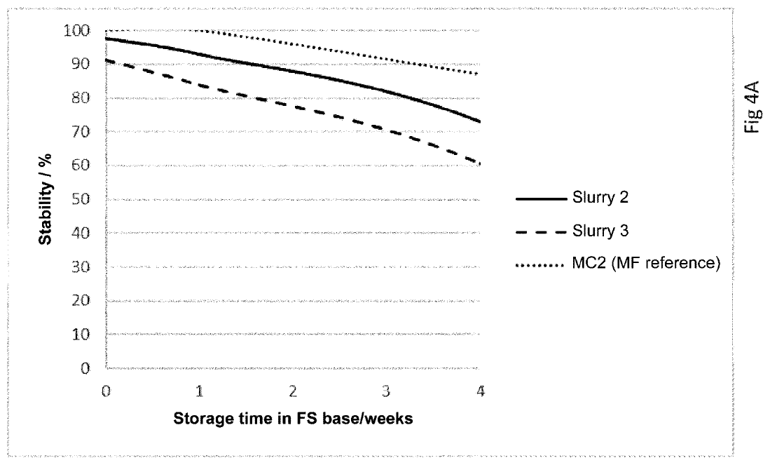

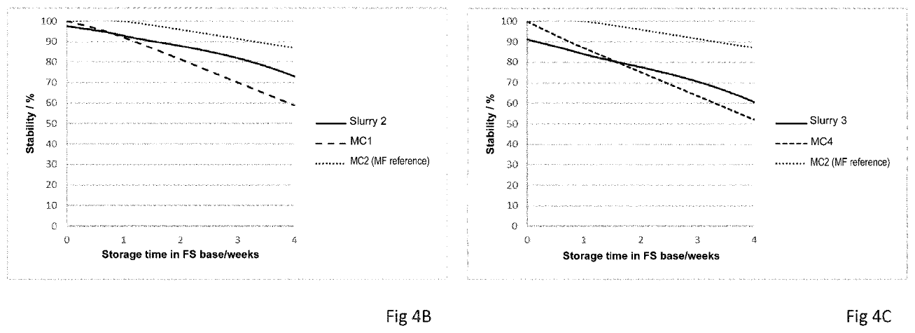

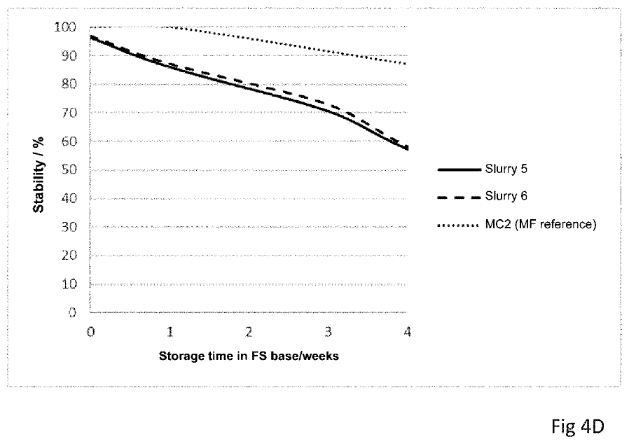

The stability course of the slurry 2 and 3 microcapsule dispersions according to the invention, as well as the MC2 reference microcapsule dispersion over 4 weeks is shown in Table 12 and FIG. 4A.

TABLE 12
Measured values of stability check 1: slurry 2, slurry 3, and MC2

| | MC2 | Slurry 2 | Slurry 3 |
|---|---|---|---|
| Run-time/weeks | Stability/% | Stability/% | Stability/% |
| 0 | 100 | 98 | 91 |
| 1 | 100 | 93 | 84 |
| 4 | 87 | 73 | 61 |

The stability course of the microcapsule dispersions slurry 5 and 6 according to the invention and the reference microcapsule dispersion MC2 over 4 weeks is shown in Table 13 and FIG. 4D.

TABLE 13
Measured values of stability check 1: slurry 5, slurry 6, and MC2

| | MC2 | Slurry 5 | Slurry 6 |
|---|---|---|---|
| Run-time/weeks | Stability/% | Stability/% | Stability/% |
| 0 | 100 | 96 | 97 |
| 1 | 100 | 86 | 87 |
| 4 | 87 | 57 | 58 |

As can be seen from Tables 12 and 13, the slurry 2, slurry 3, slurry 5, and slurry 6 microcapsules according to the invention show a stability comparable to the MF reference MC 2 after 4 weeks of storage in a model formulation. Furthermore, it is found that the microcapsules according to the invention with increased layer thickness of the stability layer slurry 2 and 3 have improved stability over a storage period of 4 weeks in comparison with the reference capsules MC1 and MC4 (cf. FIGS. 4B and 4C).

For the calculation of the capsule stability, a change in the concentration of 16 individual ingredients of the encapsulated fragrance was considered. A reduction in stability results in the encapsulated fragrance escaping, which can then be detected by gas chromatography using headspace SPME. Since all capsule dispersions were adjusted to a defined oil content of 15 wt. %, a direct comparison of the capsule samples examined is possible. Individual ingredients (or their individual signals recorded by gas chromatography) which, due to fluctuations caused by measurement technology, indicate higher concentrations than were theoretically possible in comparison with the reference standard, were only taken into account in the evaluation up to the theoretical maximum concentration.

### Example 6—Biodegradability Measurement of the Microcapsules (According to OECD 301 F)

### 6.1 General

This test is used to assess the rapid biodegradability of the microcapsules.

The standard test concentration of the samples to be examined is 1000 mg/l O2. The measuring heads and the controller measure the oxygen consumption in a closed system. Due to the consumption of oxygen and the simultaneous binding of carbon dioxide that is produced to sodium hydroxide pellets, a negative pressure is created in the system. The measuring heads register and store this pressure over the set measuring period. The stored values are read into the controller using infrared transmission. They can be transferred to a PC and evaluated using the Achat OC program.

In order to eliminate the influence of the core material on degradation, perfluorooctane was encapsulated (degradation rate=<1%).

### 6.2 Equipment and Chemicals

Equipment: OxiTop Control measuring system, from WTW incl. OxiTop OC 110 controller with interface cable for PC, 6 OxiTop C measuring heads, 6 glass bottles each with a total volume of 510 ml, 6 magnetic stirrers, 6 rubber quivers, 1 magnetic stirring system, and Achat OC readout software
   Drying cabinet ORI-BSB, set to 20° C.
   Air stones Oxygenius, 30×15×15 mm³
   Aquarium aeration pump Thomas-ASF No. 1230053
   Nutsche filter D=90 mm
   Suction flask 2 I
   White band filter MN 640 d, D=90mm, Macherey+Nagel
   “System Oxi Top Control” manual, WTW
Chemicals:
   Activated sludge from the company's own or a municipal waste water treatment plant
   Analytically pure ethylene glycol, Merck
   Reference sample with COD 1000 mg/l O2
   Walnut shell flour, Senger Naturrohstoffe
   Nutrient salt solution from the company's own or a municipal waste water
   treatment plant
   Analytically pure sodium hydroxide platelets >99%, Merck
   Cuvette test COD LCK 514, Dr. Lange

### 6.3 Procedure

### 6.3.1 Preparation of the Microcapsule Slurries

The slurry 2 and slurry 3 microcapsule dispersions were produced according to the descriptions of Examples 2 to 3, with the difference that the completely persistent perfluorooctane (degradation rate <1%) was used as the core material instead of the perfume oil. This eliminates any influence of the core material on the test result.

### 6.3.2 Sample Preparation

In the case of the extended degradation tests over 60 days, the microcapsule slurries were washed after preparation by centrifuging and redispersing in water three times in order to separate off dissolved residues. For this purpose, a sample of 20 to 30 mL is centrifuged for 10 minutes at 12,000 rpm. After sucking off the clear supernatant, 20 to 30 mL water is added and the sediment is redispersed by shaking.

### 6.3.3 Preparation of the Reference Sample

711.6 mg of ethylene glycol was dissolved in a 1 L volumetric flask and filled in up to the mark. This corresponds to a COD of 1000 mg/l O₂. Ethylene glycol is considered to be readily biodegradable and is used here as a reference.

Due to the rapid degradation of ethylene glycol, walnut shell flour was added as an additional reference for the extended 60-day test. Walnut shell flour consists of a mixture of biopolymers, particularly cellulose and lignin, and serves as a solid-based biobased reference. Due to the slow breakdown of walnut shell flour, the course of the test can be followed over the entire period of 60 days. For this purpose, 117.36 g of walnut shell flour were dispersed homogeneously in 1 l of water with stirring. Aliquots of this mixture were taken with stirring for COD determination. The required quantity was calculated based on the average COD value of 1290±33 mg/l O₂ and transferred to the OxiTop bottles while stirring.

### 6.3.4 Preparation of the Biosludge

Activated sludge was removed from the outlet of the activated sludge tank of a factory or municipal wastewater treatment plant using a 20 I bucket. After 30 minutes of settling, the supernatant water was discarded.

The concentrated biosludge in the bucket was then permanently aerated for 3 days with the help of the aquarium pump and an air stone.

### 6.3.5 Determination of the Dry Content of the Biosludge

After 3 days, 100 ml of the concentrated biosludge were filtered off using a Nutsche filter over a white band filter. The filter cake is dried in a drying cabinet at 105° C. for 24 hours.

$$T G = \frac{A}{E}$$

TG=Dry content of the biosludge in %
E=Initial weight of the filter cake in g
A=End weight of the filter cake in g
c=TG×10
c=Concentration of the biosludge in g/l
   6.3.6 Adjustment of the Samples to a COD of 1000 mg/l O₂

The COD value of the samples to be examined was determined using the COD LCK 514 cuvette test. The sample is diluted with water until the COD value of 1000 mg/l O₂ is reached.

### 6.3.7 Preparation of the Samples

6 OxiTop bottles were used for one sample, since duplicate determinations are carried out in each case.

The following measurements were carried out in 2 bottles each (duplicate determination):
Biodegradability of the sample
Biodegradability of the ethylene glycol solution (=reference solution)
Blank test (=determination of the residual degradation of the sludge itself)

Each bottle requires:
25 ml sample with a COD of 1000 mg/l O₂ (in the case of a blank sample 25 ml distilled water)
3.5 ml nutrient solution
44.5 mg o-dr (oven-dried) sludge
distilled water for filling up the bottle to a total volume of 250 ml

Using a spatula, 3 sodium hydroxide pellets were placed in each rubber quiver.

After adding the sample, nutrient solution, biosludge and distilled water to the bottles, a magnetic stir bar was placed in each bottle. Then the rubber quivers were placed on the respective bottle necks and the measuring heads screwed tightly onto the bottles.

### 6.4 Measurement and Evaluation

The programming of the OxiTop-C measuring heads and the evaluation of the data is described in detail in the “System OxiTop Control” manual from WTW.

### 6.5 Result

The degradation values of the microcapsules measured at the different times are shown in Table 14.

TABLE 14
Presentation of the degradation values according to OECD 301 (60 days)

| | Slurry 2 | | Slurry 3 | | Ethylene glycol | | |
|---|---|---|---|---|---|---|---|
| | Degradation | | Degradation | | Degradation | | Walnut shell |
| | [%] | | [%] | | [%] | | flour |
| | | Standard | | Standard | | Standard | Degradation |
| Day | Average | deviation | Average | deviation | Average | deviation | [%] |
| 0 | 0 | 0 | 0 | 0 | 0 | 0 | 0 |
| 3 | 13 | 2 | 22 | 3 | 36 | 5 | 1 |
| 5 | 32 | 6 | | | 68 | 6 | |
| 7 | 50 | 5 | 53 | 5 | 76 | 10 | 25 |
| 10 | 54 | 5 | | | 90 | 12 | |
| 14 | 53 | 9 | 75 | 8 | 95 | 7 | 38 |
| 19 | 56 | 8 | | | 99 | 2 | |
| 21 | | | 84 | 11 | | | 55 |
| 26 | 62 | 1 | | | 98 | 3 | |
| 28 | | | 86 | 10 | | | 62 |
| 32 | 76 | 1 | | | 97 | 5 | |
| 35 | 81 | 1 | 91 | 13 | 96 | 4 | 65 |
| 40 | 84 | 1 | | | 94 | 4 | |
| 42 | | | 92 | 12 | | | 66 |
| 46 | 85 | 1 | | | 88 | 3 | |
| 48 | 84 | 1 | | | 87 | 3 | |
| 49 | | | 90 | 14 | | | 66 |
| 56 | | | 91 | 13 | | | 68 |
| 60 | 74 | 3 | 91 | 13 | 81 | 2 | 69 |

The slurry 2 and slurry 3 microcapsule dispersions show very good biodegradability in the OECD301F Test. After 14 days (slurry 3), or 26 days (slurry 2), the requirements of the OECD/ECHA are met, since here a degree of degradation of >60% is present.

The degradation profile of the reference sample ethylene glycol indicates a healthy inoculum and also shows the instrumental functionality over the entire run-time of the experiment.

The wallet shell meal is characterized by the typical, step-shaped degradation profile which was expected for a complex mixing of biopolymers. Due to the continuous increase in the biol. degradability over the entire test time of 60 days, a healthy inoculum can also be deduced here.

### Example 7—Determination of the Colorfulness of the Microcapsule Dispersions

### 7.1 Experimental Description

The colorfulness of the slurry 2 and 3 microcapsule dispersions as spectral locus in the L*a*b* color space was determined by means of the following test protocol.

To determine the colorfulness of microcapsule dispersions, the portable spectrophotometer “spectro-color d/8° C. ” from Dr. Lange was used in conjunction with glass measuring cuvettes for liquids. Furthermore, the measurement took place in the associated measurement setup, which took place in the darkening of the sample during the measurement (and thus under the influence of scattered light). Before the start of the measurements, a calibration against a black and white standard (LZM268 standard set) was carried out.

The corresponding capsule dispersion is filled in undiluted form into a round glass cuvette (about 5-6 mL). By means of the associated PTFE stamp, on the one hand, the measuring range is adjusted to a defined layer thickness and any air inclusions in the dispersion are removed.

The color measurement was carried out in the context of a triple determination, wherein after each individual measurement the cuvette was rotated by about 30°. Subsequently, the average value and standard deviation are calculated.

### 7.2 Result

TABLE 15
Determination of the spectral locus of slurry 2 and 3 in
the L*a*b* color space after production and after storage

| Name | Maturity/Days | Raw data | | | | Average | SD |
|---|---|---|---|---|---|---|---|
| Slurry 3 | 0 | L | 51.76 | 52.13 | 52.17 | 52 | 0 |
| | | a | 10.94 | 10.79 | 10.65 | 11 | 0 |
| | | b | 11.67 | 10.62 | 10.68 | 11 | 0 |
| | 48 | L | 51.09 | 51.25 | 51.17 | 51 | 0 |
| | | a | 13.05 | 13.02 | 13.03 | 13 | 0 |
| | | b | 14.04 | 13.39 | 13.49 | 14 | 0 |
| Slurry 2 | 0 | L | 64.82 | 64.78 | 64.79 | 65 | 0 |
| | | a | 3.86 | 3.74 | 3.61 | 4 | 0 |
| | | b | 12.62 | 12.99 | 13.16 | 13 | 0 |
| | 48 | L | 63.82 | 63.83 | 63.76 | 64 | 0 |
| | | a | 4.35 | 4.4 | 4.38 | 4 | 0 |
| | | b | 14.27 | 13.88 | 14.23 | 14 | 0 |

### Example 8—Influence of Heat Treatment on Color Stability

In order to determine the influence of the heat treatment in the last curing step on the color stability, the production method of slurry 3 was modified. Here, the heating of the reaction mixture to 40° C. for 1 h before stirring for 14 h at room temperature was omitted. The microcapsule dispersion thus formed is referred to as slurry 3A.

Slurries 3 and 3A were prepared in parallel and, directly after, the spectral locus of samples of both microcapsule dispersions was determined according to the protocol described in Example 7. In the subsequent days 1, 2, 3, 4, and 8, samples of the slurries 3 and 3A were taken, respectively, and the spectral locus of these samples was again determined. In each case, three samples were measured per microcapsule dispersion and per day, and the average value for the three L*a*b* coordinates was determined.

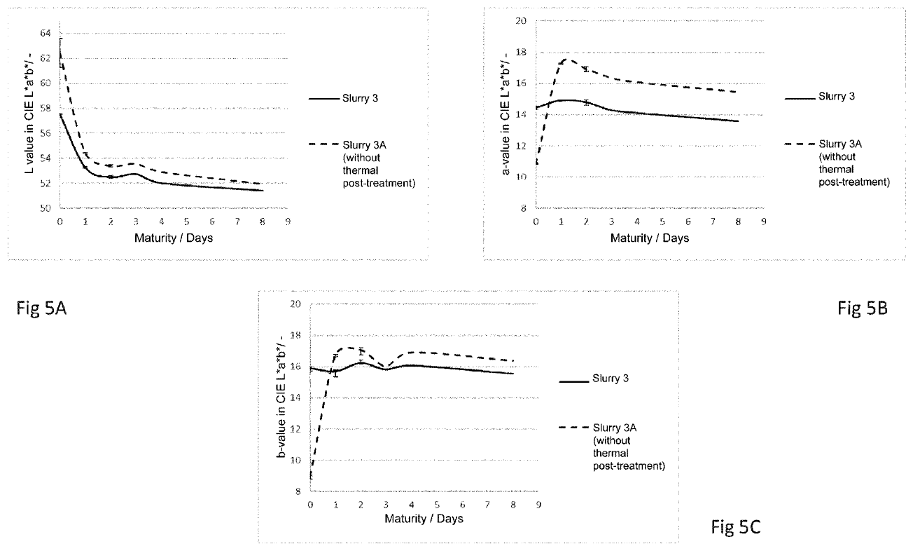

In FIG. 5 the temporal development of the L*a*b* coordinates of the two microcapsule dispersions slurry 3 and 3A is shown. The diagrams clearly show that the heat-treated microcapsule dispersion slurry 3 directly has a constant color. By contrast, in particular at the start of storage time in the case of the non-heat-treated microcapsule dispersion, a stronger change in the color with a value for ΔL* of approximately 10 occurs, for Δa* of approximately 5, and for Δb*of approximately 8.

### Example 9—Fragrance Intensity and Boost Effect

The fragrance intensity and boost effect of the capsules described herein when used in commercially available fabric softener and powder detergent formulations was examined. As a comparison, commercially available melamine-formaldehyde capsules (MF capsules) and the capsules according to PCT/EP2020/085804 were used, which have no emulsion stabilizer between the inner and outer shells. For this purpose, cotton and polyester textiles were washed with a fabric softener or full-detergent formulation without perfuming with the addition of the various perfume microcapsules (0.3 wt. % of a capsule slurry with the same capsule quantities), dried, and scored by a trained panel of experts, wherein the fragrance intensity was evaluated on a scale of 1=no fragrance to 10=very intense fragrance and the booster effect on a scale of 1=no fragrance to 5=very intense fragrance. In particular, the perfume used corresponded to the definition described herein, particularly preferably the definition for the at least one perfume composition of the core material, wherein the perfume composition, based on the total weight of all odorants contained in the perfume composition, comprises:
a) ≤10 wt. % of odorants having a CLogP of ≤2.5 and a boiling point of ≥200° C.;
b) ≥15 wt. % of at least one odorant having a CLogP of ≥4.0 and a boiling point of ≤275° C.; and
c) ≥30 wt. % of at least one odorant having a vapor pressure of ≥5 Pa at 20° C.
Washing machine: Miele Softtronic W1734 WPS
Laundry loads: 2.5 kg cotton terry towels (30×30 cm)
Washing program: detergent: main wash cycle at 40° C. (without fabric softener);
Fabric softener: main wash cycle at 40° C. with unperfumed detergent +fabric softener
Spin number: 1200 rpm
Water hardness: 12°dH
Drying type: linen
Drying conditions: 50-60% relative humidity in a clean and odorless space

The following results were obtained:

| | Scent intensity | | Booster effect | |
|---|---|---|---|---|
| Product | Cotton | Polyester | Cotton | Polyester |

Fabric softener

| | | | | |
|---|---|---|---|---|
| MF capsules (comparison) | 5 | 4 | 2 | 1.5 |
| Capsules according to | 7.5 | 7 | 4 | 3.5 |
| PCT/EP2020/085804 | | | | |
| Capsules according to the invention | 7.5 | 7 | 4 | 4 |

Powdered detergent

| | | | | |
|---|---|---|---|---|
| MF capsules (comparison) | 5 | 4 | 2 | 1.5 |
| Capsules according to | 7.5 | 7 | 4 | 3.5 |
| PCT/EP2020/085804 | | | | |
| Capsules according to the invention | 7.5 | 7 | 4 | 4 |

With regard to further advantageous embodiments of the agents and uses according to the invention, reference is made to the general part of the description and to the appended claims in order to avoid repetition.

Finally, it is expressly pointed out that the above-described embodiments of the agents and uses according to the invention are merely used to explain the claimed teaching, but do not restrict the teaching to these embodiments.

1. An agent comprising:
biodegradable microcapsules comprising:
a core material; and
a shell, wherein:
the shell comprises at least one barrier layer and a stability layer,
the barrier layer surrounds the core material,
the stability layer comprises at least one biopolymer,
the stability layer is arranged on an outer surface of the barrier layer, and
an emulsion stabilizer is arranged at the transition from the barrier layer to the stability layer.

2. The agent of claim 1, wherein the core material comprises at least one fragrance.

3. The agent of claim 1, wherein the core material comprises a perfume composition, and wherein the perfume composition, based on the total weight of all odorants contained in the perfume composition, comprises at least one of the following properties:
≤10 wt. % of all the odorants having a CLogP of ≤2.5 and a boiling point of ≥200° C.;
≥15 wt. % of all the odorants having a CLogP of ≥4.0 and a boiling point of ≤275° C.; or
<30 wt. % of all the odorants having a vapor pressure of ≥5 Pa at 20° C.

4. The agent of claim 3, wherein the perfume composition, based on the total weight of all the odorants contained in the perfume composition, comprises at least one of the following properties:
≤8 wt. % of all the odorants having a CLogP of ≤2.5 and a boiling point of ≥200° C.;
≥20 wt. % of all the odorants having a CLogP of ≥4.0 and a boiling point of ≤275° C.; or
≥40 wt. % all the odorants having a vapor pressure of ≥5 Pa at 20° C.

5. The agent of claim 1, wherein, during production of the biodegradable microcapsules, the outer surface of the barrier layer is brought into contact with the emulsion stabilizer prior to formation of the stability layer.

6. The agent of claim 1, wherein the emulsion stabilizer is a polymer or copolymer comprising at least one of the following monomers:
(1) acrylic acid derivatives of the general formula (I) 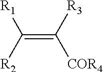
where
R₁, R₂, and R₃ are selected from hydrogen and an alkyl group having 1 to 4 carbon atoms; and
R₄ representing —OX or -NRsR₆, X representing hydrogen, an alkali metal, an ammonium group, or a C₁₋₁₈ alkyl optionally substituted by —SO₃M, or —OH, M being hydrogen, an alkali metal, or ammonium, the C₁₋₁₈ alkyl optionally substituted by —SO₃M or —OH preferably being methyl, ethyl, n-butyl, (2-)ethylhexyl, 2-sulfoethyl, or 2-sulfopropyl, R₅ and R₆ representing, independently of one another, hydrogen or a C₁₋₁₀ alkyl optionally substituted by —SO₃M, at least one of R₅ and R₆ not being hydrogen, Rs preferably being H, and R₆ preferably being 2-methyl-propan-2-yl-1-sulfonic acid;
(2) N-vinylpyrrolidone, or
(3) styrene.

7. The agent of claim 6, wherein R₄ represents —OX, X being hydrogen, C₁₋₁₀ alkyl, an alkali metal, or an ammonium group.

8. The agent of claim 1, wherein the emulsion stabilizer, based on the total weight of the biodegradable microcapsule, is in the range from 0.5 to 15.0 wt. %.

9. The agent of claim 1, wherein the barrier layer comprises at least one of an aldehyde component, an aromatic alcohol, an amine component, an acrylate component, or an isocyanate component.

10. The agent of claim 9, wherein;
the barrier layer comprises the aldehyde component selected from the group consisting of formaldehyde, glutaraldehyde, succinaldehyde, furfural, and glyoxal, and
the aldehyde component, based on the total weight of the the barrier layer, is in the range from 5 to 50 wt. %.

11. The agent of claim 9, wherein:
the barrier layer comprises the aromatic alcohol selected from the group consisting of resorcinol, phloroglucinol, and aminophenol, and
the aromatic alcohol, based on the total weight of the barrier layer, is in the range of 1.0 to 20 wt. %.

12. The agent of claim 9, wherein:
the barrier layer comprises the amine component selected from the group consisting of melamine, melamine derivatives, urea, and combinations thereof, and preferably the proportion of
the amine component, based on the total weight of the barrier layer, is in the range of 20 wt. % to 85 wt. %.

13. The agent of claim 1, wherein:
the biopolymers at least one biopolymer of the stability layer is selected from the group consisting of a protein, a polysaccharide, a phenolic macromolecule, a polyglucosamine, a polyvinyl ester, a phosphazene, and a polyester; or
the at least one biopolymer is crosslinked in the stability layer with a curing agent, and the curing agent of the stability layer is selected from the group consisting of an aldehyde, a tannin, an enzyme, an organic anhydride, an epoxy compound of a polyvalent metal cation, an amine, a polyphenol, a maleimide, a sulfide, a phenol oxide, a hydrazide, an isocyanate, an isothiocyanate, an N-hydroxysulfosuccinimide derivative, a carbodiimide derivative, and a polyol.

14. The agent of claim 1, wherein the barrier layer, based on the total weight of the shell, is at most 30 wt. %.

15. The agent aof claim 1, wherein:
the stability layer has an average thickness of at least 3 μm; and/or
the biodegradable microcapsule has a third layer which is arranged on the outside outer surface of the stability layer, wherein the third layer comprises at least one of an amine, an organic salt, and inorganic salt, an alcohol, an ether, a polyphosphazene, or a precious metal, and wherein the third layer, based on the total weight of the shell, is at most 35% wt.

16. The agent of claim 1, wherein the biodegradable microcapsules have at least one of the following features:
biodegradability of the shell measured according to OECD 301 F of at least 40%;
tightness of at most 80 wt. % of the core material used after storage for a period of 12 weeks at a temperature of 0 to 40° C.; or
a proportion of covalently bonded solids, based on the total mass of the biodegradable microcapsule wall of at least 60 wt. %.

17. The agent of claim 1, wherein the agent is a detergent, a cleaning agent, or a cosmetic agent.

18. A method for washing textiles or cleaning hard surfaces using the detergent, the cleaning agent, or the cosmetic agent of claim 17.

19. The agent of claim 2, wherein the at least one fragrance comprises a perfume oil or a perfume composition.

20. The agent of claim 6, wherein the emulsion stabilizer is the copolymer comprising 2-acrylamido-2-methylpropanesulfonic acid (AMPS).
